(19) United States
(12) Patent Application Publication
(10) Pub. No.: US 2013/0040933 A1
(43) Pub. Date: Feb. 14, 2013

(54) AZAINDOLES AS JANUS KINASE INHIBITORS

(71) Applicants: Brandon Cash, Framingham, MA (US); Christian Fischer, Natick, MA (US); Yudith Garcia, Brookline, MA (US); Joon Jung, Newton, MA (US); Jason Katz, Newton Highlands, MA (US); June Kim, West Chester, PA (US); Alexey Rivkin, Emeryville, CA (US); Adam Schell, Decatur, GA (US); Tony Siu, Brookline, MA (US); David Witter, Norfolk, MA (US); Hua Zhou, Acton, MA (US)

(72) Inventors: Brandon Cash, Framingham, MA (US); Christian Fischer, Natick, MA (US); Yudith Garcia, Brookline, MA (US); Joon Jung, Newton, MA (US); Jason Katz, Newton Highlands, MA (US); June Kim, West Chester, PA (US); Alexey Rivkin, Emeryville, CA (US); Adam Schell, Decatur, GA (US); Tony Siu, Brookline, MA (US); David Witter, Norfolk, MA (US); Hua Zhou, Acton, MA (US)

(21) Appl. No.: 13/643,323

(22) PCT Filed: Apr. 21, 2011
(86) PCT No.: PCT/US11/33394

§ 371 Date: Oct. 25, 2012

Related U.S. Application Data
(60) Provisional application No. 61/328,276, filed on Apr. 27, 2010.

(51) Int. Cl. A61K 31/497; C07D 519/00; A61K 31/5377; A61K 31/541; A61K 31/4985; A61P 35/00; A61P 11/06; A61P 37/00; A61P 1/04; A61P 25/00; A61P 3/10; C07D 471/04; A61P 19/02

(52) U.S. Cl. 514/210.18; 544/405; 544/357; 544/120; 544/58.2; 544/350; 544/295; 514/255.05; 514/252.11; 514/234.5; 540/524; 514/212.08; 514/228.2; 514/249

(57) ABSTRACT

The present invention provides compounds of formula I: or a pharmaceutically acceptable salt thereof. Compounds of formula I are inhibitors of Janus kinases and as such are useful for the treatment of various diseases and conditions mediated by said enzymes, such as rheumatoid arthritis, asthma, COPD, ALS, and the like.

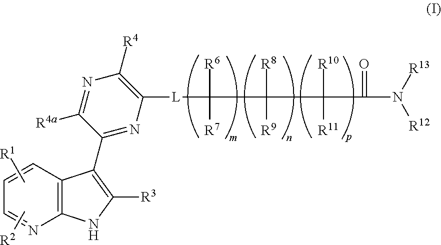

### BACKGROUND OF THE INVENTION

Studies of interferon (IFN)-induced receptor mediated gene expression led to the initial discovery of a Janus kinase (Jak) signaling pathway, which has been shown to be a common signaling pathway used by many cytokines and growth factors. The mammalian Jak family of intracellular tyrosine kinases, has four members; Jak1, Jak2, Jak3 and Tyk2. Jaks range in size from 120 to 140 kDa's and contain seven conserved Jak homology (JH) domains which define this kinase super family.

Prototypically, the binding of a cytokine to its cell surface receptor results in receptor dimerization and subsequent activation/phosphorylation of Jak tyrosine kinases which are constitutively associated with the receptor. Specific tyrosine residues on the receptor are then phosphorylated by activated Jak's and serve as docking sites for a family of latent cytoplasmic transcription factors known as Signal Transducers and Activators of Transcription (STATS). STATS are phosphorylated by Jaks, dimerize, then translocate to the nucleus where they bind specific DNA elements and activate gene transcription.

Many pro-inflammatory cytokines (IL-6, IL-12, IL-15, IL-23, GM-CSF and IFN-γ) which are implicated in autoimmune diseases mediate their activity through the Jak kinases. As a consequence, these enzymes have long been considered attractive drug targets. The essential role of Jaks in mediating the biological effects of cytokines has been confirmed by natural mutations in humans and targeted disruption in mice. Humans with a genetic loss of Jak3 have a severe combined immunodefiency (SCID) phenotype due to a developmental block in T and NK cell development and nonfunctional B-cells. Humans lacking Tyk2 are susceptible to microbial infection, have a Th2 bias with Hyper-IgE syndrome and defective cytokine signaling (IL-6, 10, 12 and 23). Signaling can be restored by transfection of the wild type kinase.

Animal KO models of the JAK family of kinases have demonstrated significant phenotypes. Jak1 KO animals exhibit defective responses to class 2 cytokines (IL-10 family), those utilizing the common gamma chain γ_{c} (IL-2, IL-4 etc) and gp130 receptor subunits (IL-6, LIF, OSM), resulting in perinatal lethality due to developmental, neurological and lymphoid defects. Jak2 KO mice exhibit defective erythropoiesis caused by a block in EPO signaling, resulting in embryonic lethality. Jak3 KO mice are viable but exhibit a SCID phenotype with nonfunctional T-cells and a lack of B and NK-cells (similar to human mutation). Tyk2 KO animals manifest modest viral susceptibility, reduced IL-12 responses, resistance to arthritis and enhanced Th2 cell-mediated allergic inflammation.

A considerable body of literature has accumulated that link the JAK/STAT pathway to various diseases and disorders including hyperproliferative disorders and cancer such as leukemia and lymphomas, immunological and inflammatory disorders such as transplant rejection, asthma, chronic obstructive pulmonary disease, allergies, rheumatoid arthritis, type I diabetes, amyotropic lateral sclerosis and multiple sclerosis.

### SUMMARY OF THE INVENTION

The present invention provides novel compounds which are inhibitors of JAKs. The invention also provides a method for the treatment and prevention of JAK-mediated diseases and disorders using the novel compounds, as well as pharmaceutical compositions containing the compounds.

### DETAILED DESCRIPTION OF THE INVENTION

The present invention provides compounds of formula I:

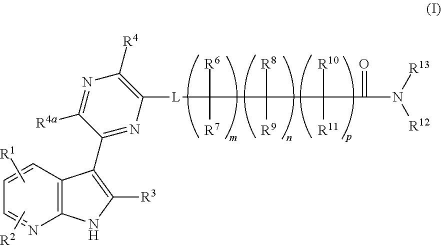

and pharmaceutically acceptable salts thereof; wherein
L is a bond, phenylene, O or NR⁵;
m is 0 or 1; n is 0, 1 or 2; p is 0 or 1; with the proviso that when L is O or NR⁵ m+n+p is 1 to 4;
R¹ and R² are each independently selected from the group consisting of: (1) H, (2) hydroxy, (3) cyano, (4) halogen, (5) C₁₋₆alkyl, C₂₋₁₀alkenyl, C₂₋₁₀alkynyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano, NR^{b}R^{c}, NHC(O)C₁₋₆alkyl and S(O)₂C₁₋₆alkyl, (6) CO₂R^{a}, (7) nitro, (8) NR^{b}R^{c}, (9) CONR^{b}R^{c}, (10) NR^{b}(CONR^{b}R^{c}), (11) S(O)₂NR^{b}R^{c} and (12) a ring containing moiety which is: aryl, aryloxy, arylC₁₋₆alkyl, arylcarbonyl, cycloalkyl, a heterocycle, a heteroaryl, any of which ring is optionally substituted with 1 to 5 groups independently selected from R^{z};
R³ is H or C₁₋₃alkyl;
R⁴ and R^{4a} are each independently selected from the group consisting of (1) hydrogen, (2) hydroxy, (3) cyano, (4) halogen, (5) C₁₋₆alkyl, C₂₋₁₀alkenyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano and NR^{b}R^{c}, (6) CO₂R^{a}, (7) nitro, (8) NR^{b}R^{c}, and (9) CONR^{b}R^{c}; or
when L is NR⁵, R⁴ and R⁵ together complete a pyrrole ring;
R⁵-R¹³ are each independently selected from the group consisting of (1) H, (2) C₁₋₆alkyl optionally substituted with 1 to 5 groups independently selected from halogen, CN, OR^{a}, CO₂R^{a}, CONR^{b}R^{c}, NR^{b}R^{c}, C₃₋₆cycloalkyl, optionally substituted aryl and optionally substituted heteroaryl wherein the optional substituents for aryl and heteroaryl are 1 to 3 groups independently selected from OR^{a}, NR^{b}R^{c}, halogen, C₁₋₆alkyl and C₁₋₆haloalkyl, (3) C₃₋₆cycloalkyl and (4) 2-azepinone; or
R⁶ and R¹⁰ together complete a cycloalkyl, cycloalkenyl or heterocyclyl, each of which is optionally substituted with 1 to 5 groups independently selected from R^{y}; or
R⁸ and R⁹ together complete a cycloalkyl optionally substituted with 1 to 5 groups independently selected from R^{y}; or
when L is NO, R⁵ and R¹⁰ together complete a heterocycle optionally substituted with 1 to 5 groups independently selected from R^{y}; or
when n is 2, two R⁸ groups on adjacent carbon atoms together form a bond; or two R⁸ groups and two R⁹ groups on adjacent carbon atoms together complete a cycloalkenyl, aryl or heteroaryl each or which is optionally substituted with 1 to 4 groups independently selected from hydroxy, cyano, halogen, NR^{b}R^{c}, C₁₋₆haloalkyl, C₁₋₆hydroxyalkyl, C₂₋₁₀alkenyl, C₁₋₆alkylcarbonyl, C₁₋₆alkoxy, C₁₋₆haloalkoxy and C₁₋₆alkoxycarbonyl; or
R¹², R¹³ and the nitrogen atom to which they are attached together form a 4- to 7-membered ring optionally having an additional heteroatom selected from NR^{x}, O and S(O)_{q}, and said ring is optionally substituted with 1 to 4 halogen atoms or a group selected from CO₂R^{a} and CONR^{a}R^{b};
R^{a} is H or C₁₋₆alkyl;
R^{b} and R^{c} are each independently selected from H, C₁₋₆alkyl, C₁₋₆haloalkyl, C₃₋₆cycloalkyl and C₁₋₆hydroxyalkyl; or
R^{b}, R^{c} and the nitrogen atom to which they are attached together form a 4- to 7-membered ring optionally having an additional heteroatom selected from NR^{x}, O and S(O)_{q}, said ring being optionally substituted with 1 to 4 halogen atoms;
R^{x} is selected from the group consisting of H, C₁₋₆alkyl, C₁₋₆haloalkyl, C₁₋₆alkylcarbonyl and S(O)₂C₁₋₆alkyl;
R^{y} is selected from the group consisting of (1) hydroxy, (2) oxo, (3) cyano, (4) halogen, (5) C₁₋₆alkyl, C₂₋₁₀alkenyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano, NR^{b}R^{c}, NHC(O)C₁₋₆alkyl and S(O)₂C₁₋₆alkyl, (6) CO₂R^{a}, (7) nitro, (8) NR^{b}R^{c}, (9) CONR^{b}R^{c}, (10) NR^{b}(CONR^{b}R^{c}), and (11) S(O)₂NR^{b}R^{c};
R^{z} is selected from the group consisting of (1) hydroxy, (2) oxo, (3) cyano, (4) halogen, (5) C₁₋₆alkyl, C₂₋₁₀alkenyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano, NR^{b}R^{c}, NHC(O)C₁₋₆alkyl and S(O)₂C₁₋₆alkyl, (6) CO₂R^{a}, (7) nitro, (8) NR^{b}R^{c}, (9) CONR^{b}R^{c}, (10) NR^{b}(CONR^{b}R^{c}), (11) S(O)₂NR^{b}R^{c} and (12) a ring containing moiety which is: C₆₋₁₀aryl, C₆₋₁₀arylC₁₋₆alkyl, cycloalkyl, azetidinyl, a 5 or 6 membered saturated or partially saturated heterocyclic ring containing 1, 2 or 3 heteroatoms independently selected from N, O and S, a 5 membered heteroaromatic ring containing 1, 2, 3 or 4 heteroatoms independently selected from N, O and S, not more than one heteroatom of which is O or S, a 6 membered heteroaromatic ring containing 1, 2 or 3 nitrogen atoms or a 7-15 membered unsaturated, partially saturated or saturated heterocyclic ring containing 1, 2, 3 or 4 heteroatoms independently selected from N, O and S; any of which rings being optionally substituted 1 to 5 groups independently selected from the group consisting of hydroxy, cyano, halogen, C₁₋₆alkyl, C₁₋₆alkoxy, C₂₋₁₀alkenyl, C₁₋₆haloalkyl, amino, C₁₋₆alkylamino and di(C₁₋₆alkyl)amino.

In one group of formula I are compounds wherein L is NR⁵.

In another group of formula I are compounds wherein L is O.

In another group of formula I are compounds wherein L is a bond or phenylene. In one subset thereof m, n and p are each 0; in another subset thereof, m and p are each 0, and n is 1 or 2.

In another group of formula I are compounds wherein m and p are each 0, and n is 1 or 2. In one subset thereof each R⁸ and R⁹ is independently selected from H and C₁₋₃alkyl. In another subset R⁸ and R⁹ on the same carbon atom together form a C₃₋₆cycloalkyl. In another subset, n is 2 and two R⁸ groups on adjacent carbon atoms together form a bond (i.e., —(R⁹)C═C(R⁹)—). In another subset the two R⁸ and two R⁹ groups on adjacent carbon atoms together form a cycloalkenyl, aryl or heteroaryl group (i.e., a ring moiety

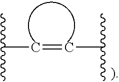 [SMILES: *C=C*]

In another group of formula I are compounds wherein R⁶ and R¹⁰ together complete a cycloalkyl, cycloalkenyl or heterocyclyl (i.e., a ring moiety

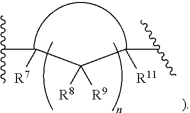

In another group of formula I are compounds wherein one of R¹² and R¹³ is H and the other is —CH₂CF₃.

In another group of formula I are compounds having the formula Ia:

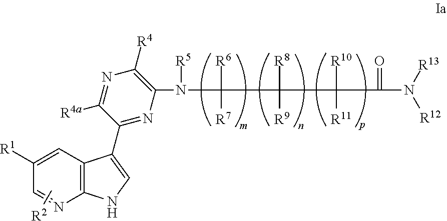

or a pharmaceutically acceptable salt thereof, wherein the variables are as defined under formula I.

In one subset of formula Ia are compounds wherein in and p are each 0, and n is 1 or 2. In one embodiment thereof n is 1 and one of R⁸ and R⁹ is C₁₋₄alkyl, and the other is H or C₁₋₄alkyl; examples are where one of R⁸ and R⁹ is methyl or isopropyl, and the other is H, and where R⁸ and R⁹ are each methyl. In another embodiment thereof n is 1 and R⁸ and R⁹ together complete a C₃₋₆cycloalkyl; for example —C(R⁸)(R⁹)— may be 1,1-cyclopentanediyl. In another embodiment thereof n is 2 and two R⁸ groups on adjacent carbon atoms together form a bond. In another subset thereof n is 2 and the two R⁸ and two R⁹ groups on adjacent carbon atoms together form a cycloalkenyl, aryl or heteroaryl group (i.e., a ring moiety

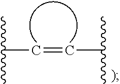 [SMILES: *C=C*]

examples or said ring moiety include 1,2-cyclopentenediyl, 1,2-phenylene, 5,6-benzothiophenediyl.

In another subset of formula Ia are compounds wherein R⁶ and R¹⁰ together complete a cycloalkyl, cycloalkenyl or heterocyclyl (i.e., a ring moiety

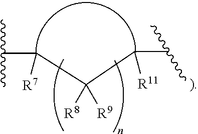

In one embodiment thereof are compounds wherein said ring moiety is a cycloalkyl group; examples of cycloalkyl include 1,3-cyclopentanediyl, 1,4-cyclohexanediyl, 2,3-norbornanediyl, 7,8-spiro[4.5]decanediyl, 5,6-perhydroindandiyl and 5,6,7,8-tetrahydronaphthalene-6,7-diyl.

In another subset of formula Ia are compounds wherein R⁵ and R¹⁰ together complete a heterocycle optionally substituted with one or two groups independently selected from halogen, OR^{a} and C₁₋₃alkyl. In one embodiment thereof said heterocycle is 4- to 6-membered ring optionally having an additional heteroatom selected from N—R^{b}, O and S(O)_{q} wherein q is 0, 1 or 2; examples of such rings include azetidine, pyrrolidine, piperidine, morpholine, thiamorpholine and piperazine. In another embodiment such heterocycle is bicyclic such as 5,6,7,8-tetrahydroimidazo[1,2-a]pyrazine.

In another subset of formula Ia are compounds wherein one of R¹² and R¹³ is H and the other is —CH₂CF₃.

In another subset of formula Ia are compounds wherein R⁴ and R^{4a} are each H.

In another group of formula I are compounds of formula Ib:

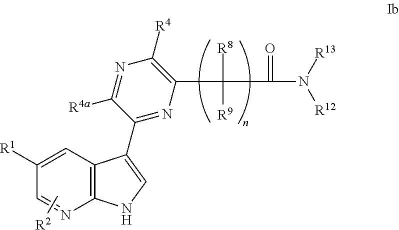

or a pharmaceutically acceptable salt thereof, wherein the variables are as defined under formula I. In one subset of formula Ib are compounds wherein n is 0. In another subset of formula Ib are compounds wherein n is 1. In another subset of formula Ib are compounds wherein n is 2; in one embodiment thereof are compounds wherein R⁸ and R⁹ are independently selected from H and C₁₋₃alkyl; in another embodiment thereof are compounds where two R⁸ groups on adjacent carbon atoms together form a bond. In another subset of formula Ia are compounds wherein one of R¹² and R¹³ is H and the other is —CH₂CF₃.

In another group of formula I are compounds of formula Ic:

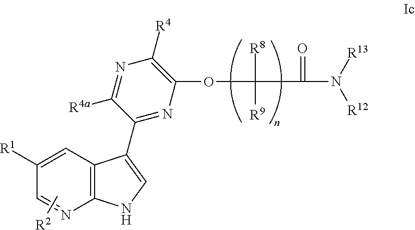

or a pharmaceutically acceptable salt thereof, wherein n is 1 or 2 and the other variables are as defined under formula I. In one subset are compounds wherein n is 1; in one embodiment thereof R⁸ and R⁹ are each selected from H and C₁₋₃alkyl; in another embodiment R⁸ and R⁹ together complete a 3- to 6-membered cycloalkyl.

In the above formulas I, Ia, Ib and Ic, examples of R¹ include, but are not limited to halogen, such as chlorine and fluorine; C₃₋₁₀cycloalkyl, such as cyclopropyl; C₁₋₆alkyl, such as methyl; optionally substituted C₂₋₁₀alkenyl, such as 3-(dimethylamino)-1-propenyl; optionally substituted C₂₋₁₀alkynyl, such as 3-methyoxy-1-propynyl; optionally substituted heteroaryl, such as 1-methylpyrazolyl, 2-cyanomethylpyridyl, 2-aminopyrimidinyl, methoxy-pyridyl, cyanopyridyl, fluoropyridyl; optionally substituted phenyl, such as pyrrolidin-1-ylphenyl, morpholin-4-ylphenyl, 1-hydroxy-2,2,2-trifluoroethylphenyl, (1-pyrrolidin-1-ylethyl)phenyl, morpholin-4-ylmethylphenyl, dimethylaminomethylphenyl, trifluoromethoxyphenyl; optionally substituted heterocycle, such as 5,6-dihydropyrrolo[1,2-b]pyrazolyl, dihydropyranyl; NR^{b}R^{c}, such as 3-fluoro-1-pyrrolidinyl; carboxy; alkyl carboxylate; carboxamide; and cyano. R¹ is preferably attached to the 5-position of 1H-pyrrolo[2,3-b]pyridine, and R² is preferably H.

In the above formulas I, Ia, Ib and Ic, examples of R¹² include, but are not limited to, ethyl, 2,2,2-trifluoroethyl, cyanomethyl, 2-Cl-3-pyridylmethyl, 5-methyl-3-isoxazolylmethyl, 2-(4-pyrazolyl)ethyl, 2-cyclopropylethyl, 3-(3-pyridyl)-2-propyl, 2-thienylmethyl, dimethyl-aminocarbonylmethyl, 2-oxo-3-azepanyl, 4-aminophenylmethyl, 2-(2-thienyl)ethyl, cyclopentyl, cyclobutyl, 1-hydroxy-3-(4-hydroxyphenyl)-2-propyl, 3-(1,1-dioxido-4-thiamoipholinyl)propyl, (4-trifluoromethylphenyl)methyl, 3,3,3-trifluoropropyl, 2-methylpropyl and 2,2-dimethylpropyl. R13 is preferably H or C₁₋₆alkyl. Examples of cyclic NR¹²R¹³ include, but are not limited to, morpholinyl, 3,3-difluoropyrrolidinyl, 3,3,4,4-tetrafluoropyrrolidinyl, 4,4-difluoropiperidinyl and 4-(2,2,2-trifluoroethyl)piperazinyl.

Representative compounds of the instant invention include:
N²-[6-(5-chloro-1H-pyrrolo[2,3-b]pyridin-3-yl)pyrazin-2-yl]-N-(2,2,2-trifluoroethyl)-D-alaninamide;
(1R,3R)-3{-[6-(5-chloro-1H-pyrrolo[2,3-b]pyridin-3-yl)pyrazin-2-yl]amino}-N-(2,2,2-trifluoro-ethyl)cyclopentanecarboxamide;
(1S,3R)-3-{[6-(5-chloro-1H-pyrrolo[2,3-b]pyridin-3-yl)pyrazin-2-yl]amino}-N-(2,2,2-trifluoro-ethyl)cyclopentanecarboxamide;
(1R,3S)-3-{[6-(5-chloro-1,1-pyrrolo[2,3-b]pyridin-3-yl)pyrazin-2-yl]amino}-N-(2,2,2-trifluoro-ethyl)cyclopentanecarboxamide;
(1R,3S)-3-{[6-(5-chloro-1H-pyrrolo[2,3-b]pyridin-3-yl)pyrazin-2-yl]amino}-N-(2,2,2-trifluoro-ethyl)cyclopentanecarboxamide;
1-[6-(5-chloro-1H-pyrrolo[2,3-b]pyridin-3-yl)pyrazin-2-yl]-N-(2,2,2-trifluoroethyl)-L-prolinamide;
1-[6-(5-chloro-1H-pyrrolo[2,3-b]pyridin-3-yl)pyrazin-2-yl]-N-(2,2,2-trifluoroethyl)-D-prolinamide;
1-[6-(5-chloro-1H-pyrrolo[2,3-b]pyridin-3-yl)pyrazin-2-yl]-N-ethyl-D-prolinamide;
1-[6-(5-chloro-1H-pyrrolo[2,3-b]pyridin-3-yl)pyrazin-2-yl]-4,4-difluoro-N-(2,2,2-trifluoroethyl)-L-prolinamide;
1-[6-(5-chloro-1H-pyrrolo[2,3-b]pyridin-3-yl)pyrazin-2-yl]-2-methyl-N-(2,2,2-trifluoro-ethyl)azetidine-2-carboxamide;
(2S)-1-[6-(5-chloro-1H-pyrrolo[2,3-b]pyridin-3-yl)pyrazin-2-yl]-2-methyl-N-(2,2,2-trifluoro-ethyl)azetidine-2-carboxamide;
(2R)-1-[6-(5-chloro-1H-pyrrolo[2,3-b]pyridin-3-yl)pyrazin-2-yl]-2-methyl-N-(2,2,2-trifluoro-ethyl)azetidine-2-carboxamide;
N²-[6-(5-chloro-1H-pyrrolo[2,3-b]pyridin-3-yl)pyrazin-2-yl]-N-(2,2,2-trifluoroethyl)-D-valinamide;
tert-butyl (3R)-4-[6-(5-chloro-1H-pynolo[2,3-b]pyridin-3-yl)pyrazin-2-yl]-3-[(2,2,2-trifluoro-ethyl)carbamoyl]piperazine-1-carboxylate;
N²-[6-(5-chloro-1H-pyrrolo[2,3-b]pyridin-3-yl)pyrazin-2-yl]-2-methyl-N-(2,2,2-trifluoro-ethyl)alaninamide;
1-[6-(5-chloro-1H-pyrrolo[2,3-b]pyridin-3-yl)pyrazin-2-yl]-N-(2,2,2-trifluoroethyl)azetidine-3-carboxamide;
methyl 3-(5-chloro-1H-pyrrolo[2,3-b]pyridin-3-yl)-5-({(1S)-1-methyl-2-oxo-2-[(2,2,2-trifluoro-ethyl)amino]ethyl}amino)pyrazine-2-carboxylate;
N²-[6-(5-Chloro-1H-pyrrolo[2,3-b]pyridin-3-yl)pyrazin-2-yl]-N-(2,2,2-trifluoroethyl)-1,-alaninamide;
N²-[6-(5-chloro-1H-pyrrolo[2,3-b]pyridin-3-yl)pyrazin-2-yl]-N-(cyanomethyl)-L-alaninamide;
(2S,3S)-1-[6-(5-chloro-1H-pyrrolo[2,3-b]pyridin-3-yl)pyrazin-2-yl]-3-hydroxy-N-(2,2,2-trifluoroethyl)azetidine-2-carboxamide;
6-(5-chloro-1H-pyrrolo[2,3-b]pyridin-3-yl)-N-[(1S)-1-methyl-2-morpholin-4-yl-2-oxoethyl]-pyrazin-2-amine;
6-(5-chloro-1H-pyrrolo[2,3-b]pyridin-3-yl)-N-[(1S)-2-(3,3-difluoropyrrolidin-1-yl)-1-methyl-2-oxoethyl]pyrazin-2-amine;
6-(5-chloro-1H-pyrrolo[2,3-b]pyridin-3-yl)-N-[(1S)-1-methyl-2-oxo-2-(3,3,4,4-tetrafluoro-pyrrolidin-1-yl)ethyl]pyrazin-2-amine;
N-[(2-chloropyridin-3-yl)methyl]-N²-[6-(5-chloro-1H-pyrrolo[2,3-b]pyridin-3-yl)pyrazin-2-yl]-D-alaninamide;
N²-[6-(5-chloro-1H-pyrrolo[2,3-b]pyridin-3-yl)pyrazin-2-yl]-N-[(5-methylisoxazol-3-yl)methyl]-D-alaninamide;
N²-[6-(5-chloro-1H-pyrrolo[2,3-b]pyridin-3-yl)pyrazin-2-yl]-N-[2-(1H-pyrazol-4-yl)ethyl]-D-alaninamide;
N²-[6-(5-chloro-1H-pyrrolo[2,3-b]pyridin-3-yl)pyrazin-2-yl]-N-(2-cyclopropylethyl)-D-alaninamide;
N²-[6-(5-chloro-1H-pyrrolo[2,3-b]pyridin-3-yl)pyrazin-2-yl]-N-(1-methyl-2-pyridin-3-ylethyl)-D-alaninamide;
N²-[6-(5-chloro-1H-pyrrolo[2,3-b]pyridin-3-yl)pyrazin-2-yl]-N-(thiophen-2-ylmethyl)-D-alaninamide;
N-[6-(5-chloro-1H-1-pyrrolo[2,3-b]pyridin-3-yl)pyrazin-2-yl]-D-alanyl-N,N-dimethylglycinamide;
6-(5-chloro-1H-pyrrolo[2,3-b]pyridin-3-yl)-N-[(1R)-2-(4,4-difluoropiperidin-1-yl)-1-methyl-2-oxoethyl]pyrazin-2-amine;
N²-[6-(5-chloro-1H-pyrrolo[2,3-b]pyridin-3-yl)pyrazin-2-yl]-N-(2-oxoazepan-3-yl)-D-alaninamide;
N-(4-aminobenzyl)-N²-[6-(5-chloro-1H-pyrrolo[2,3-b]pyridin-3-yl)pyrazin-2-yl]-D-alaninamide;
N²-[6-(5-chloro-1H-pyrrolo[2,3-b]pyridin-3-yl)pyrazin-2-yl]-N-(2-thiophen-2-ylethyl)-D-alaninamide;
N-[6-(5-chloro-1H-pyrrolo[2,3-b]pyridin-3-yl)pyrazin-2-yl]-D-alanyl-N-cyclopentylglycinamide;
N²-[6-(5-chloro-1H-pyrrolo[2,3-b]pyridin-3-yl)pyrazin-2-yl]-N-cyclobutyl-D-alaninamide;
N²-[6-(5-chloro-1H-pyrrolo[2,3-b]pyridin-3-yl)pyrazin-2-yl]-N-[(1S)-2-hydroxy-1-(4-hydroxybenzyl)ethyl]-D-alaninamide;
6-(5-chloro-1H-pyrrolo[2,3-b]pyridin-3-yl)-N-{(1R)-1-methyl-2-oxo-2-[4-(2,2,2-trifluoro-ethyl)piperazin-1-yl]ethyl}pyrazin-2-amine;
N²-[6-(5-chloro-1H-pyrrolo[2,3-b]pyridin-3-yl)pyrazin-2-yl]-N-[3-(1,1-dioxidothiomorpholin-4-yl)propyl]-D-alaninamide;
N²-[6-(5-chloro-1H-1-pyrrolo[2,3-b]pyridin-3-yl)pyrazin-2-yl]-N-[4-(trifluoromethyl)benzyl]-D-alaninamide;
N²-[6-(5-chloro-1H-pyrrolo[2,3-b]pyridin-3-yl)pyrazin-2-yl]-N-(3,3,3-trifluoropropyl)-L-alaninamide;
N²-[6-(5-chloro-1H-pyrrolo[2,3-b]pyridin-3-yl)pyrazin-2-yl]-N-(2-methylpropyl)-L-alaninamide;
N²-[6-(5-chloro-1H-pyrrolo[2,3-b]pyridin-3-yl)pyrazin-2-yl]-N-(2,2-dimethylpropyl)-L-alaninamide;
1-{[6-(5-chloro-1H-pyrrolo[2,3-b]pyridin-3-yl)pyrazin-2-yl]amino}-N-(2,2,2-trifluoro-ethyl)cyclopentanecarboxamide;
1-{[6-(5-chloro-1H-pyrrolo[2,3-b]pyridin-3-yl)pyrazin-2-yl]amino}-N-(3,3,3-trifluoro-propyl)cyclopentanecarboxamide;
(2S,3S)-1 [6-(5-Chloro-1H-pyrrolo[2,3-b]pyridin-3-yl)pyrazin-2-yl]-3-hydroxy-N-(2,2,2-trifluoroethyl)azetidine-2-carboxamide;
(2R)-1-[6-(5-Chloro-1H-pyrrolo[2,3-b]pyridin-3-yl)pyrazin-2-yl]-4-methylsulfonyl-N-(2,2,2-trifluoroethyl)piperazine-2-carboxamide;
(2R)-1-[6-(5-chloro-1H-pyrrolo[2,3-b]pyridin-3-yl)pyrazin-2-yl]-N-(2,2,2-trifluoroethyl)-piperazine-2-carboxamide;
(2R)-4-acetyl-1-[6-(5-chloro-1H-pyrrolo[2,3-b]pyridin-3-yl)pyrazin-2-yl]-N-(2,2,2-trifluoro-ethyl)piperazine-2-carboxamide;
(2R)-1-[6-(5-chloro-1H-pyrrolo[2,3-b]pyridin-3-yl)pyrazin-2-yl]-4-(2,2-dimethylpropanoyl)-N-(2,2,2-trifluoroethyl)piperazine-2-carboxamide;
2-[3-(5-chloro-1H-pyrrolo[2,3-b]pyridin-3-yl)-5H-pyrrolo[2,3-b]pyrazin-5-yl]-N-(2,2,2-trifluoro-ethyl)acetamide;
1-{[6-(5-chloro-1H-pyrrolo[2,3-b]pyridin-3-yl)pyrazin-2-yl]oxy}-N-(2,2,2-trifluoroethyl)-cyclopropanecarboxamide;
1-{[6-(5-chloro-1H-pyrrolo[2,3-b]pyridin-3-yl)pyrazin-2-yl]oxy}cyclopropanecarboxamide;
1-{[6-(5-chloro-1H-pyrrolo[2,3-b]pyridin-3-yl)pyrazin-2-yl]oxy}-N,N-dimethylcyclopropane-carboxamide;
1-{[6-(5-chloro-1H-pyrrolo[2,3-b]pyridin-3-yl)pyrazin-2-yl]oxy}-trifluoropropyl)cyclo-propanecarboxamide;
2(S)2-{[6-(5-chloro-1H-pyrrolo[2,3-b]pyridin-3-yl)pyrazin-2-yl]oxy}-N-(2,2,2-trifluoroethyl)-propanamide;
6-(5-chloro-1H-pyrrolo[2,3-b]pyridin-3-yl)-N-(2,2,2-trifluoroethyl)pyrazine-2-carboxamide;
(2R)-1-{[6-(5-chloro-1H-pyrrolo[2,3-b]pyridin-3-yl)pyrazin-2-yl]carbonyl}-N-(2,2,2-trifluoro-ethyl)piperazine-2-carboxamide;
2-[6-(5-chloro-1H-pyrrolo[2,3-b]pyridin-3-yl)pyrazin-2-yl]-N-(2,2,2-trifluoroethyl)acetamide;
5-chloro-3-[6-(2-morpholin-4-yl-2-oxoethyl)pyrazin-2-yl]-1H-pyrrolo[2,3-b]pyridine;
2-[6-(5-chloro-1H-pyrrolo[2,3-b]pyridin-3-yl)pyrazin-2-yl]-N-(2-cyanoethyl)acetamide;
2-[6-(5-chloro-1H-pyrrolo[2,3-b]pyridin-3-yl)pyrazin-2-yl]-N-(3,3,3-trifluoropropyl)acetamide;
3-{6-[2-(4-acetylpiperazin-1-yl)-2-oxoethyl]pyrazin-2-yl}-5-chloro-1H-pyrrolo[2,3-b]pyridine;
2-[6-(5-chloro-1H-pyrrolo[2,3-b]pyridin-3-yl)pyrazin-2-yl]-N-(cyanomethyl)acetamide;
(2E)-3-[6-(5-chloro-1H-pyrrolo[2,3-b]pyridin-3-yl)pyrazin-2-yl]-2-methyl-N-(2,2,2-trifluoro-ethyl)prop-2-enamide;
4-[6-(5-chloro-1H-pyrrolo[2,3-b]pyridin-3-yl)pyrazin-2-yl]-N-(2,2,2-trifluoroethyl)benzamide;
2-[6-(5-chloro-1H-pyrrolo[2,3-b]pyridin-3-yl)pyrazin-2-yl]-N-(2,2,2-trifluoroethyl)cyclopent-1-ene-1-carboxamide;
2-[6-(5-chloro-1H-pyrrolo[2,3-b]pyridin-3-yl)pyrazin-2-yl]-1-methyl-N-(2,2,2-trifluoroethyl)-cyclopropanecarboxamide;
3-[6-(5-chloro-1H-pyrrolo[2,3-b]pyridin-3-yl)pyrazin-2-yl]-2-methyl-N-(2,2,2-trifluoroethyl)-propanamide;
3-[6-(5-chloro-1H-pyrrolo[2,3-b]pyridin-3-yl)pyrazin-2-yl]-N-(2,2,2-trifluoroethyl)propanamide;
N²-[6-(5-cyclopropyl-1H-pyrrolo[2,3-b]pyridin-3-yl)pyrazin-2-yl]-N²-(2,2,2-trifluoroethyl)-D-alaninamide;
N²-{6-[5-(1-methyl-1H-pyrazol-4-yl)-1H-pyrrolo[2,3-b]pyridin-3-yl]pyrazin-2-yl}-N-(2,2,2-trifluoroethyl)-D-alaninamide;
N²-[6-(5-methyl-1H-1-pyrrolo[2,3-b]pyridin-3-yl)pyrazin-2-yl]-N-(2,2,2-trifluoroethyl)-D-alaninamide;
N²-{6-[5-(3-pyrrolidin-1-ylphenyl)-1H-pyrrolo[2,3-b]pyridin-3-yl]pyrazin-2-yl}-N-(2,2,2-trifluoroethyl)-D-alaninamide;
N²-(6-{5-[6-(cyanomethyl)pyridin-3-yl}-1H-pyrrolo[2,3-b]pyridin-3-yl]pyrazin-2-yl)-N-(2,2,2-trifluoroethyl)-D-alaninamide;
N-(2,2,2-trifluoroethyl)-N²-(6-{5-[4-(2,2,2-trifluoro-1-hydroxyethyl)phenyl]-1H-1-pyrrolo[2,3-b]pyridin-3-yl}pyrazin-2-yl)-D-alaninamide;
N²-(6-{5-[(1E)-3-(dimethylamino)prop-1-en-1-yl]-1H-pyrrolo[2,3-b]pyridin-3-yl}pyrazin-2-yl)-N-(2,2,2-trifluoroethyl)-D-alaninamide;
N²-{6-[5-(1-methyl-1H-pyrazol-5-yl)-1H-pyrrolo[2,3-b]pyridin-3-yl]pyrazin-2-yl}-N-(2,2,2-trifluoroethyl)-D-alaninamide;
N²-{6-[5-(2-aminopyrimidin-5-yl)-1H-pyrrolo[2,3-b]pyridin-3-yl]pyrazin-2-yl}-N-(2,2,2-trifluoroethyl)-D-alaninamide;
N²-{6-[5-(3-methoxyprop-1-yn-1-yl)-1H-pyrrolo[2,3-b]pyridin-3-yl]pyrazin-2-yl}-N-(2,2,2-trifluoroethyl)-D-alaninamide;
N²-(6-{5-[(3-fluoropyrrolidin-1-yl)methyl}-1H-pyrrolo[2,3-b]pyridin-3-yl]pyrazin-2-yl)-N-(2,2,2-trifluoroethyl)-D-alaninamide;
N²-[6-(5-cyclopropyl-1H-pyrrolo[2,3-b]pyridin-3-yl)pyrazin-2-yl]-2-methyl-N-(2,2,2-trifluoro-ethyl)alaninamide;
N²-{6-[5-(5,6-dihydro-4H-pyrrolo[1,2-b]pyrazol-3-yl)-1H-pyrrolo[2,3-b]pyridin-3-yl]pyrazin-2-yl}-N-(2,2,2-trifluoroethyl)-D-alaninamide;
N²-{6-[5-(3-morpholin-4-ylphenyl)-1H-pyrrolo[2,3-b]pyridin-3-yl]pyrazin-2-yl}-N-(2,2,2-trifluoroethyl)-D-alaninamide;
N²-(6-{5-[3-(1-pyrrolidin-1-ylethyl)phenyl]-1H-pyrrolo[2,3-b]pyridin-3-yl}pyrazin-2-yl)-N-(2,2,2-trifluoroethyl)-D-alaninamide;
N²-(6-{5-[3-(morpholin-4-ylmethyl)phenyl]-1H-pyrrolo[2,3-b]pyridin-3-yl}pyrazin-2-yl)-N-(2,2,2-trifluoroethyl)-D-alaninamide;
N²-[6-(5-{2-[(dimethylamino)methyl]phenyl}1H-pyrrolo[2,3-b]pyridin-3-yl)pyrazin-2-yl]-N-(2,2,2-trifluoroethyl)-D-alaninamide;
N-(2,2,2-trifluoroethyl)-N²-(6-{5-[2-(trifluoromethoxy)phenyl]-1H-pyrrolo[2,3-b]pyridin-3-yl}pyrazin-2-yl)-D-alaninamide;
N²-{6-[5-(3,6-dihydro-2H-pyran-4-yl)-1H-pyrrolo[2,3-b]pyridin-3-yl]pyrazin-2-yl}-N-(2,2,2-trifluoroethyl)-D-alaninamide;
N²-{6-[5-(5,6-dihydro-2H-pyran-3-yl)-1H-pyrrolo[2,3-h]pyridin-3-yl]pyrazin-2-yl}trifluoroethyl)-D-alaninamide;
N²-{6-[5-(4-pyrrolidin-1-ylphenyl)-1H-pyrrolo[2,3-b]pyridin-3-yl]pyrazin-2-yl}-N-(2,2,2-trifluoroethyl)-D-alaninamide;
N²-{6-[5-(5-methoxypyridin-3-yl)-1H-pyrrolo[2,3-b]pyridin-3-yl]pyrazin-2-yl}-N-(2,2,2-trifluoroethyl)-D-alaninamide;
N²-{6-[5-(5-cyanopyridin-3-yl)-1H-pyrrolo[2,3-b]pyridin-3-yl]pyrazin-2-yl}-N-(2,2,2-trifluoroethyl)-D-alaninamide;
N²-{6-[5-(2-fluoropyridin-3-yl)-1H-pyrrolo[2,3-b]pyridin-3-yl]pyrazin-2-yl}-N-(2,2,2-trifluoroethyl)-D-alaninamide;
3-[6-({(2R)-3-methyl-1-oxo-1-[(2,2,2-trifluoroethyl)amino]butan-2-yl}amino)pyrazin-2-yl]-1H-pyrrolo[2,3-b]pyridine-5-carboxylic acid;
3-[6-({(2R)-3-methyl-1-oxo-1-[(2,2,2-trifluoroethyl)amino]butan-2-yl}amino)pyrazin-2-yl]-1H-pyrrolo[2,3-b]pyridine-5-carboxamide;
N²-[6-(5-fluoro-1H-pyrrolo[2,3-b]pyridin-3-yl)pyrazin-2-yl]-N-(2,2,2-trifluoroethyl)-D-valinamide;
N²-[6-(5-cyano-1-pyrrolo[2,3-b]pyridin-3-yl)pyrazin-2-yl]-N-(2,2,2-trifluoroethyl)-D-valinamide;
Methyl 3-[6-({(2R)-3-methyl-1-oxo-1-[(2,2,2-trifluoroethyl)amino]butan-2-yl}amino)pyrazin-2-yl]-1H-pyrrolo[2,3-b]pyridine-5-carboxylate;
N²-[6-(4-chloro-1H-pyrrolo[2,3-b]pyridin-3-yl)pyrazin-2-yl]-N-(2,2,2-trifluoroethyl)-D-valinamide;
N²-[6-(5-fluoro-1H-pyrrolo[2,3-b]pyridin-3-yl)pyrazin-2-yl]-2-methyl-N-(2,2,2-trifluoroethyl)-alaninamide.

The invention also encompasses pharmaceutical compositions containing a compound of formula I, and methods for treatment or prevention of JAK mediated diseases using compounds of formula I.

The invention is described using the following definitions unless otherwise indicated.

The term “alkyl” refers to linear or branched alkyl chains having the indicated number of carbon atoms. Non-limiting examples of alkyl groups include methyl, ethyl, propyl, isopropyl, butyl, s- and t-butyl, pentyl, hexyl, and the like.

“Alkoxy” means a linear or branched alkyl chain having the indicated number of carbon atoms attached through an oxygen atom. C₁₋₆Alkoxy, for example, includes methoxy, ethoxy, propoxy, isopropoxy, and the like.

The term “cycloalkyl” means a carbocyclic ring system having 3 to 12 ring carbon atoms; said ring system may be (a) a monocyclic saturated carbocycle optionally fused to a benzene or a partially unsaturated carbocycle, or (b) a bicyclic saturated carbocycle. For a bicyclic system, within either (a) or (b), the rings are fused across two adjacent ring carbon atoms (e.g., decalin), at one ring carbon atom (e.g., spiro[2.2]pentane), or are bridged groups (e.g., norbornane). Additional examples within the above meaning include, but are not limited to, cyclopropane, cyclobutane, cyclopentane, cyclohexane, perhydroindan, tetrahydronaphthalene, spiro[4.5]decane, bicyclo[2.2.2]octane, and the like.

“Alkenyl” refers to a straight or branched hydrocarbon chain containing at least one carbon to carbon double bond. Preferably one carbon to carbon double bond is present, and up to four nonaromatic carbon-carbon double bonds may be present. Examples of alkenyl groups include ethenyl, propenyl, butenyl and 2-methylbutenyl.

“Alkylcarbonyl” or “alkoxycarbonyl” denotes an alkyl or alkoxy radical, respectively, attached via a carbonyl (C═O) radical. Suitable examples of alkylcarbonyl groups include methylcarbonyl, ethylcarbonyl, propylcarbonyl, isopropylcarbonyl and tert-butylcarbonyl. Examples of alkoxycarbonyl include methoxycarbonyl, ethoxycarbonyl, propoxycarbonyl, isopropoxycarbonyl and tert-butoxycarbonyl. The term “C₆₋₁₀arylcarbonyl” can be construed analogously, and an example of this group is benzoyl.

“Aryl” means a 6-10 membered monocyclic or bicyclic carbon only ring systems in which at least one ring is aromatic. Examples include phenyl, tetrahydronaphthyl, indanyl, dihydronaphthyl and naphthyl.

The term “halogen” or “halo” includes F, Cl, Br, and I.

“Haloalkyl” or “haloalkoxy” means an alkyl or an alkoxy group as described above, respectively, wherein one or more (in particular 1 to 5) hydrogen atoms have been replaced by halogen atoms, with up to complete substitution of all hydrogen atoms with halo groups. C₁₋₆haloalkyl, for example, includes —CF₃, —CF₂CF₃, CHFCH₃, and the like.

“Hydroxyalkyl” means an alkyl group as described above in which one or more (in particular 1 to 3) hydrogen atoms have been replaced by hydroxy groups. Examples include CH₂OH, CH₂CHOH and CHOHCH₃.

The term “heterocycle” or “heterocyclic” represents a monocyclic or bicyclic 4-10 membered ring system in which at least one ring is non-aromatic (saturated or partially unsaturated) and containing at least one heteroatom selected from O, S and N. In a bicyclic ring system, the second ring may be a heteroaryl, heterocycle or a saturated, partially unsaturated or aromatic carbocycle, and the point(s) of attachment to the rest of the molecule may be on either ring. Examples of heterocycle include, but are not limited to, azetidinyl, pyrrolidinyl, piperidinyl, piperazinyl, morpholinyl, thiamorpholinyl, tetrahydrofuranyl, dihydrofuranyl, tetrahydrothienyl, tetrahydropyranyl, dihydropyranyl, dihydroimidazolyl, dihydroindolyl, 5,6,7,8-tetrahydroimidazo[1,2-a]pyrazine and the like.

The teen “heteroaryl” or “heteroaromatic” (Het) as used herein represents a 5-10 membered aromatic ring system containing one ring (monocyclic) or two fused rings (bicyclic), and 1-4 heteroatoms independently selected from O, S and N. For a bicyclic heteroaryl only one of the rings need to be heteroaromatic, the second ring may be a heteroaromatic or an aromatic, saturated, or partially unsatuated carbocycle, and the point(s) of attachment to the rest of the molecule may be on either ring. Het includes, but is not limited to, furanyl, thienyl, pyrrolyl, imidazolyl, pyrazolyl, oxazolyl, thiazolyl, isoxazolyl, isothiazolyl, triazolyl, oxadiazolyl, thiadiazolyl, tetrazolyl, pyridyl, pyrimidinyl, pyrazinyl, pyridazinyl, triazinyl, quinolinyl, isoquinolinyl, naphthyridinyl, benzothienyl, benzofuranyl, indolyl, isoindolyl, indolizinyl, indazolyl, purinyl, quinolizinyl, phthalazinyl, quinoxalinyl, quinazolinyl, benzoxazolyl, benzisoxazolyl, 5,6,7,8-tetrahydroquinolinyl, 5,6-dihydropyrrolo[1,2-b]pyrazolyl, pyrrolopyridine, furopyridine and thienopyridine.

“Therapeutically effective amount” means that amount of a drug or pharmaceutical agent that will elicit the biological or medical response of a tissue, a system, animal or human that is being sought by a researcher, veterinarian, medical doctor or other clinician.

The term “treatment” or “treating” includes alleviating, ameliorating, relieving or otherwise reducing the signs and symptoms associated with a disease or disorder.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of formula I, and pharmaceutically acceptable excipients.

The term “optionally substituted” means “unsubstituted or substituted,” and therefore, the generic structural formulas described herein encompasses compounds containing the specified optional substituent as well as compounds that do not contain the optional substituent.

Each variable is independently defined each time it occurs within the generic structural formula definitions. For example, when there is more than one substituent for aryl/heteroaryl, each substituent is independently selected at each occurrence, and each substituent can be the same or different from the other(s). As another example, for the group —(CR⁸R⁹)₂—, each occurrence of the two R⁸ groups may be the same or different. As used herein, unless explicitly stated to the contrary, each reference to a specific compound of the present invention or a generic formula of compounds of the present invention is intended to include the compound(s) as well as pharmaceutically acceptable salts thereof.

### Optical Isomers-Diastereomers-Geometric Isomers-Tautomers

Compounds of formula I contain one or more asymmetric centers and can thus occur as racemates and racemic mixtures, single enantiomers, diastereomeric mixtures and individual diastereomers. The present invention is meant to comprehend all such isomeric forms of the compounds of formula I, either as single species or mixtures thereof.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of formula I.

Compounds of the formula I may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of a compound of the general formula I may be obtained by stereospecific synthesis using optically pure starting materials, intermediates or reagents of known configuration.

### Salts

The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, unless otherwise specified, references to the compound of formula I, Ia, Ib, Ic and Id, subsets thereof, embodiments thereof, as well as specific compounds are meant to also include the pharmaceutically acceptable salts.

Furthermore, some of the crystalline forms for compounds of the present invention may exist as polymorphs and as such all forms are intended to be included in the present invention. In addition, some of the compounds of the instant invention may form solvates with water (hydrates) or common organic solvents. Such solvates are encompassed within the scope of this invention.

### Labelled Compounds

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

### Utilities

Compounds of formula I are inhibitors of JAK, and some compounds may preferentially inhibit one member of the JAK family over other members; for example a compound of formula I may preferentially or selectively inhibit JAK3. Compounds of formula I or pharmaceutically acceptable salts thereof and pharmaceutical compositions containing them can be used to treat or prevent a variety of conditions or diseases mediated by Janus kinases, in particular diseases or conditions that can be ameliorated by the inhibition of a Janus kinase such as JAK1, JAK2 or JAK3. Such conditions and diseases include, but are not limited to: (1) arthritis, including rheumatoid arthritis, juvenile arthritis, and psoriatic arthritis; (2) asthma and other obstructive airways diseases, including chronic asthma, late asthma, airway hyper-responsiveness, bronchitis, bronchial asthma, allergic asthma, intrinsic asthma, extrinsic asthma, dust asthma, recurrent airway obstruction, and chronic obstruction pulmonary disease including emphysema; (3) autoimmune diseases or disorders, including those designated as single organ or single cell-type autoimmune disorders, for example Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, chronic aggressive hepatitis, ulcerative colitis and membranous glomerulopathy, those designated as involving systemic autoimmune disorder, for example systemic lupus erythematosis, rheumatoid arthritis, Sjogren's syndrome, Reiter's syndrome, polymyositis-dermatomyositis, systemic sclerosis, polyarteritis nodosa, multiple sclerosis and bullous pemphigoid, and additional autoimmune diseases, which can be B-cell (humoral) based or T-cell based, including Cogan's syndrome, ankylosing spondylitis, Wegener's granulomatosis, autoimmune alopecia, Type I or juvenile onset diabetes, and thyroiditis; (4) cancers or tumors, including alimentary/gastro-intestinal tract cancer, colon cancer, liver cancer, skin cancer including mast cell tumor and squamous cell carcinoma, breast and mammary cancer, ovarian cancer, prostate cancer, lymphoma, leukemia, including acute myelogenous leukemia and chronic myelogenous leukemia, kidney cancer, lung cancer, muscle cancer, bone cancer, bladder cancer, brain cancer, melanoma including oral and metastatic melanoma, Kaposi's sarcoma, myelomas including multiple myeloma, myeloproliferative disorders, proliferative diabetic retinopathy, and angiogenic-associated disorders including solid tumors; (5) diabetes, including Type I diabetes and complications from diabetes; (6) eye diseases, disorders or conditions including autoimmune diseases of the eye, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization; (7) intestinal inflammations, allergies or conditions including Crohn's disease and/or ulcerative colitis, inflammatory bowel disease, coeliac diseases, proctitis, eosinophilic gastroenteritis, and mastocytosis; (8) neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemica, renal ischemia, heart attacks, cardiac hypertrophy, atherosclerosis and arteriosclerosis, organ hypoxia, and platelet aggregation; (9) skin diseases, conditions or disorders including atopic dermatitis, eczema, psoriasis, scleroderma, pruritus and other pruritic conditions; (10) allergic reactions including anaphylaxis, allergic rhinitis, allergic dermatitis, allergic urticaria, angioedema, allergic asthma, or allergic reaction to insect bites, food, drugs, or pollen; (11) transplant rejection, including pancreas islet transplant rejection, bone marrow transplant rejection, graft-versus-host disease, organ and cell transplant rejection such as bone marrow, cartilage, cornea, heart, intervertebral disc, islet, kidney, limb, liver, lung, muscle, myoblast, nerve, pancreas, skin, small intestine, or trachea, and xeno transplantation.

Accordingly, another aspect of the present invention provides a method for the treatment or prevention of a JAK-mediated disease or disorder (particularly JAK-3 mediated) comprising administering to a mammal in need thereof a therapeutically effective amount of a compound of formula I. In one embodiment such disease include asthma and rheumatoid arthritis.

Another aspect of the present invention provides for the use of a compound of formula I in the manufacture of a medicament for the treatment or prevention of a JAK-mediated diseases or disorder.

### Dose Ranges

The magnitude of prophylactic or therapeutic dose of a compound of formula I will, of course, vary with the nature and the severity of the condition to be treated and with the particular compound of formula I and its route of administration. It will also vary according to a variety of factors including the age, weight, general health, sex, diet, time of administration, rate of excretion, drug combination and response of the individual patient. In general, the daily dose from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for the oral administration of humans may contain from 0.05 mg to 5 g of active agent compounded with an appropriate and convenient amount of carrier material which may vary from about 5 to about 99.95 percent of the total composition. Dosage unit forms will generally contain between from about 0.1 mg to about 0.4 g of an active ingredient, typically 0.5 mg, 1 mg, 2 mg, 5 mg, 10 mg, 25 mg, 50 mg, 100 mg, 200 mg, or 400 mg.

### Pharmaceutical Compositions

Another aspect of the present invention provides pharmaceutical compositions comprising a compound of formula I with a pharmaceutically acceptable carrier. For the treatment of any of the prostanoid mediated diseases compounds of formula I may be administered orally, by inhalation spray, topically, parenterally or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Dosage forms for inhaled administration may conveniently be formulated as aerosols or dry powders. For compositions suitable and/or adapted for inhaled administration, it is preferred that the active substance is in a particle-size-reduced form, and more preferably the size-reduced form is obtained or obtainable by micronization.

In one embodiment the medicinal preparation is adapted for use with a pressurized metered dose inhaler (pMDI) which releases a metered dose of medicine upon each actuation. The formulation for pMDIs can be in the form of solutions or suspensions in halogenated hydrocarbon propellants. The type of propellant being used in pMDIs is being shifted to hydrofluoroalkanes (HFAs), also known as hydrofluorocarbons (HFCs). In particular, 1,1,1,2-tetrafluoroethane (HFA 134a) and 1,1,1,2,3,3,3-heptafluoropropane (HFA 227) are used in several currently marketed pharmaceutical inhalation products. The composition may include other pharmaceutically acceptable excipients for inhalation use such as ethanol, oleic acid, polyvinylpyrrolidone and the like.

Pressurized MDIs typically have two components. Firstly, there is a canister component in which the drug particles are stored under pressure in a suspension or solution form. Secondly, there is a receptacle component used to hold and actuate the canister. Typically, a canister will contain multiple doses of the formulation, although it is possible to have single dose canisters as well. The canister component typically includes a valve outlet from which the contents of the canister can be discharged. Aerosol medication is dispensed from the pMDI by applying a force on the canister component to push it into the receptacle component thereby opening the valve outlet and causing the medication particles to be conveyed from the valve outlet through the receptacle component and discharged from an outlet of the receptacle. Upon discharge from the canister, the medication particles are “atomized”, forming an aerosol. It is intended that the patient coordinate the discharge of aerosolized medication with his or her inhalation, so that the medication particles are entrained in the patient's aspiratory flow and conveyed to the lungs. Typically, pMDIs use propellants to pressurize the contents of the canister and to propel the medication particles out of the outlet of the receptacle component. In pMDIs, the formulation is provided in a liquid or suspension form, and resides within the container along with the propellant. The propellant can take a variety of forms. For example, the propellant can comprise a compressed gas or liquefied gas.

In another embodiment the medicinal preparation is adapted for use with a dry powder inhaler (DPI). The inhalation composition suitable for use in DPIs typically comprises particles of the active ingredient and particles of a pharmaceutically acceptable carrier. The particle size of the active material may vary from about 0.1 μm to about 10 μm; however, for effective delivery to the distal lung, at least 95 percent of the active agent particles are 5 μm or smaller. Each of the active agent can be present in a concentration of 0.01-99%. Typically however, each of the active agents is present in a concentration of about 0.05 to 50%, more typically about 0.2-20% of the total weight of the composition.

As noted above, in addition to the active ingredients, the inhalable powder preferably includes pharmaceutically acceptable carrier, which may be composed of any pharmacologically inert material or combination of materials which is acceptable for inhalation. Advantageously, the carrier particles are composed of one or more crystalline sugars; the carrier particles may be composed of one or more sugar alcohols or polyols. Preferably, the carrier particles are particles of dextrose or lactose, especially lactose. In embodiments of the present invention which utilize conventional dry powder inhalers, such as the Handihaler, Rotohaler, Diskhaler, Twisthaler and Turbohaler, the particle size of the carrier particles may range from about 10 microns to about 1000 microns. In certain of these embodiments, the particle size of the carrier particles may range from about 20 microns to about 120 microns. In certain other embodiments, the size of at least 90% by weight of the carrier particles is less than 1000 microns and preferably lies between 60 microns and 1000 microns. The relatively large size of these carrier particles gives good flow and entrainment characteristics. Where present, the amount of carrier particles will generally be up to 95%, for example, up to 90%, advantageously up to 80% and preferably up to 50% by weight based on the total weight of the powder. The amount of any fine excipient material, if present, may be up to 50% and advantageously up to 30%, especially up to 20%, by weight, based on the total weight of the powder. The powder may optionally contain a performance modifier such as L-leucine or another amino acid, and/or metals salts of stearic acid such as magnesium or calcium stearate.

Compounds of formula I may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing the compound of formula I are employed. (For purposes of this application, topical application shall include mouth washes and gargles.) Topical formulations may generally be comprised of a pharmaceutical carrier, cosolvent, emulsifier, penetration enhancer, preservative system, and emollient.

Combinations with Other Drugs

For the treatment and prevention of JAK mediated diseases, compound of formula I may be co-administered with other therapeutic agents. Thus in another aspect the present invention provides pharmaceutical compositions for treating JAK mediated diseases comprising a therapeutically effective amount of a compound of formula I and one or more other therapeutic agents. In particular, for the treatment of the inflammatory diseases rheumatoid arthritis, psoriasis, inflammatory bowel disease, COPD, asthma and allergic rhinitis a compound of formula I may be combined with agents such as: (1) TNF-α inhibitors such as Remicade® and Enbrel®); (2) non-selective COX-I/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin); (3) COX-2 inhibitors (such as meloxicam, celecoxib, rofecoxib, valdecoxib and etoricoxib); (4) other agents for treatment of rheumatoid arthritis including low dose methotrexate, lefunomide, ciclesonide, hydroxychloroquine, d-penicillamine, auranofin or parenteral or oral gold; (5) leukotriene biosynthesis inhibitor, 5-lipoxygenase (5-LO) inhibitor or 5-lipoxygenase activating protein (FLAP) antagonist such as zileuton; (6) LTD4 receptor antagonist such as zafirlukast, montelukast and pranlukast; (7) PDE4 inhibitor such as roflumilast; (8) antihistaminic H1 receptor antagonists such as cetirizine, loratadine, desloratadine, fexofenadine, astemizole, azelastine, and chlorpheniramine; (9) α1- and α2-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride; (10) anticholinergic agents such as ipratropium bromide, tiotropium bromide, oxitropium bromide, aclindinium bromide, glycopyrrolate, pirenzepine, and telenzepine; (11) β-adrenoceptor agonists such as metaproterenol, isoproterenol, isoprenaline, albuterol, salbutamol, formoterol, salmeterol, terbutaline, orciprenaline, bitolterol mesylate, and pirbuterol, or methylxanthanines including theophylline and aminophylline, sodium cromoglycate; (12) insulin-like growth factor type I (IGF-1) mimetic; (13) inhaled glucocorticoid with reduced systemic side effects, such as prednisone, prednisolone, flunisolide, triamcinolone acetonide, beclomethasone dipropionate, budesonide, fluticasone propionate, ciclesonide and mometasone furoate.

### Methods of Synthesis

Compounds of formula I of the present invention can be prepared according to the synthetic routes outlined in the following scheme(s) and by following the methods described herein. Abbreviations used herein include: Ac=acetyl; APCI=atmospheric pressure chemical ionization; BOC=t-butoxycarbonyl; CBZ=benzyloxycarbonyl; DABCO=1,4-diaza-bicyclo[2.2.2]octane; DCM=dichloromethane; DIEA=diisopropylethylamine; DMF=dimethylformamide; DMSO=dimethyl sulfoxide; EDC=N-ethyl-N-(3-dimethylaminopropyl)-carbodiimide; ES=electrospray; Et=ethyl; EtOAc=ethyl acetate; HOBT=1-hydroxybenzotriazole; HPLC=high pressure liquid chromatography; IPA=isopropyl alcohol; iPr=isopropyl; Me=methyl; NMP=N-methylpyrrolidone; NPLC=normal phase liquid chromatography; n-Pr=n-propyl; PdCl₂(dppf)=[1,1′-bis(diphenylphosphino)ferrocene]-dichloropalladium(II); PE=petroleum ether; Ph=phenyl; Py-BOP=(benzotriazol-1-yloxy)-tripyrrolidinophosphonium hexafluorophosphate; RP=reversed-phase; RT, rt=room temperature; TEA=triethylamine; TFA=trifluoroacetic acid; THF tetrahydrofuran; Ts=toluenesulfonyl or tosyl.

Scheme 1 depicts the construction of compounds of formula I using the Suzuki coupling reaction. N-protected pyrrolopyridine boron derivative (1) is reacted with an amide substituted pyrazine halide (2) in the presence of a palladium catalyst to provide, after removal of the N-protecting group, a compound of formula I. Examples of suitable boron derivative include K⁺BF₃— and pinacolboranyl, and an example of suitable N-protecting group is toluenesulfonyl, which can be removed by a base such as NaOH. Alternatively, compounds of formula I may be similarly prepared from boron derivative (1) and an ester substituted pyrazine halide (3); the resultant ester product is converted to the corresponding carboxylic acid, which undergoes peptide coupling reaction with an appropriate amine to provide, after removal of the N-protecting group, a compound of formula I.

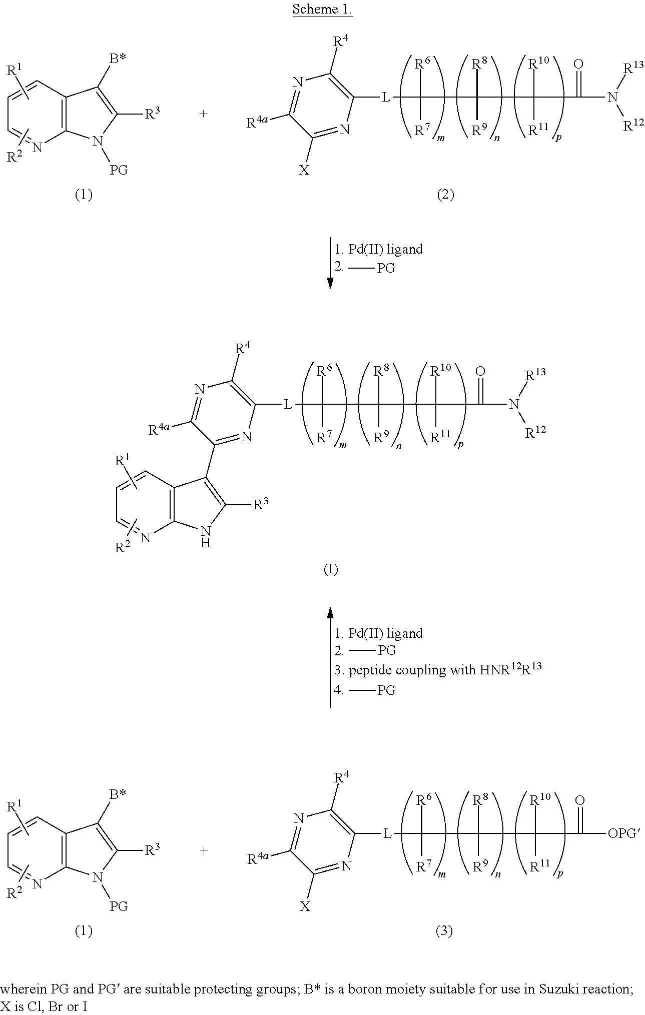

Pyrazine halides (2) and (3) in which L is an oxygen or NR5 can be assembled as depicted in Scheme 2. A pyrazine dihalide (4) is reacted with an amine or alcohol (5) under nucleophilic substitution conditions to provide the corresponding coupled product 2a/3a.

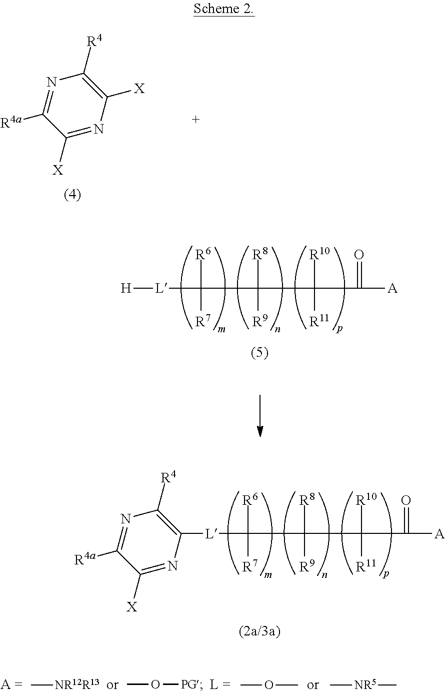

Pyrazine halides (2) and (3) in which L is phenylene or a bond may be prepared as shown in Scheme 3. Pyrazine dihalide (4) is reacted with a boron derivative (6) under Suzuki coupling conditions to afford the product 2b/3b.

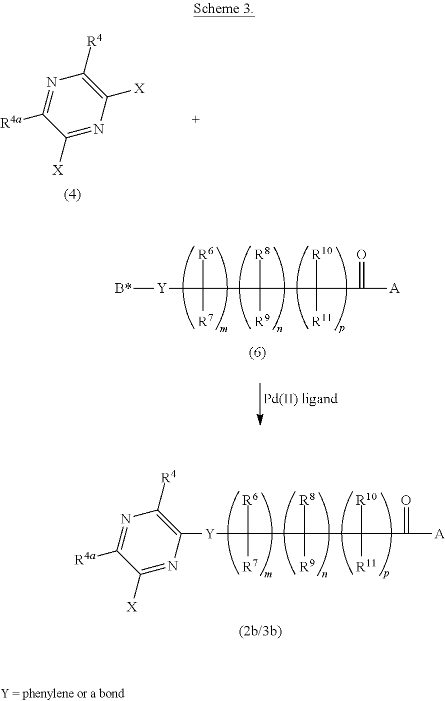

Scheme 4 depicts the preparation of a group of compounds of formula I-1. Starting from pyrazine dihalide (4) and a malonic acid diester (7), the base-catalyzed nucleophilic displacement produces the pyrazine halide diester (3c). Suzuki coupling of (1) and (3c) followed by saponification produces the acid (8), which provides a compound of formula I-1 after peptide coupling with HNR¹²R¹³ and removal of the N-protecting group.

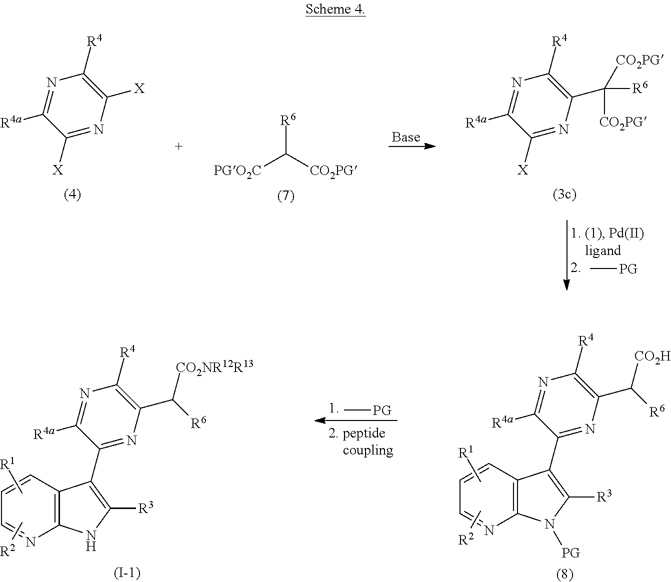

Scheme 5 depicts the preparation of a group of compounds of formula I-2. Starting from carbonyl substituted pyrazine halide (9) and a Wittig reagent (10), the olefin substituted pyrazine halide (3d) is produced. Saponification of (3d) and the subsequent peptide coupling of the resultant acid with HNR¹²R¹³ affords the amide (10), which provides a compound of formula I-2 after Suzuki coupling with the boron derivative (1) and removal of the N-protecting group.

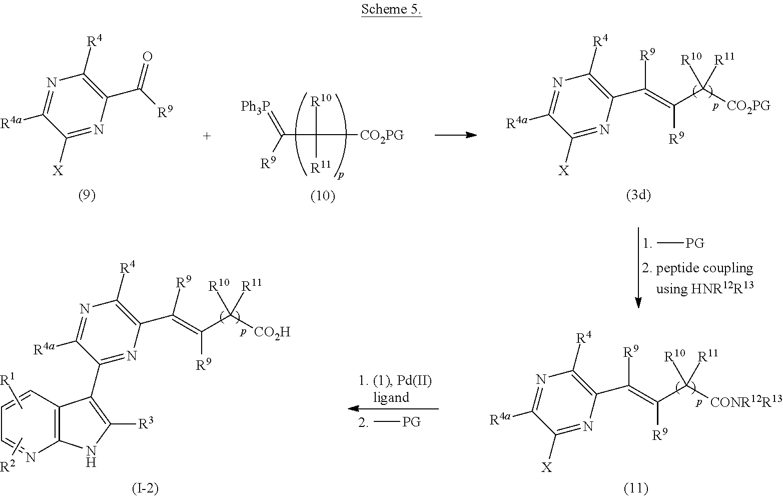

Scheme 6 depicts the preparation of a group of compounds of formula I-3. Starting from pyrazine dihalide (4) and an organic zinc halide (12) under Negishi cross-coupling conditions, the alkyl substituted pyrazine halide (3e) is produced. Suzuki coupling with (1), removal of the protecting groups, and peptide coupling with HNR¹²R¹³ affords a compound of formula I-3.

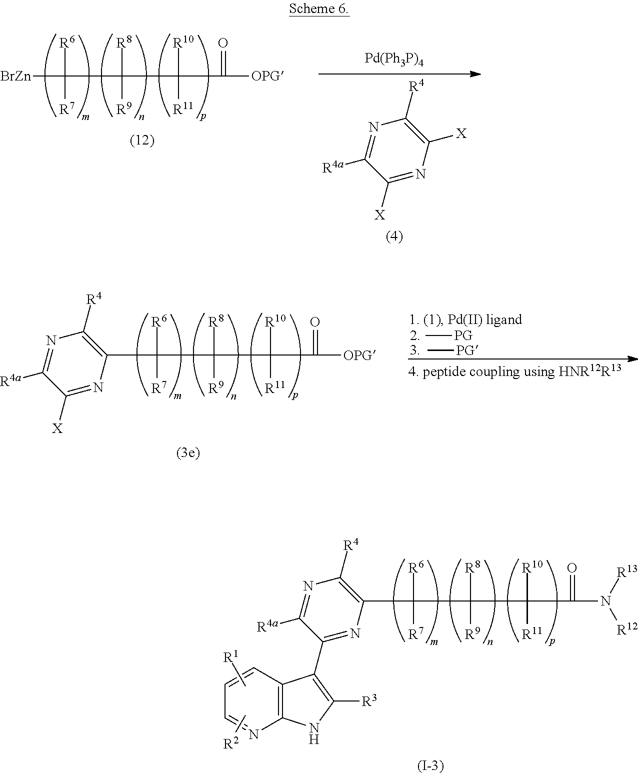

Scheme 7 shows that a compound in which R¹ is a halogen can be further elaborated using Suzuki coupling reaction to provide a compound (13) in which R^{1a} is a moiety that can be introduced using a Suzuki reagent, such as alkyl, alkenyl, cycloalkyl, aryl, heteroaryl and heterocycle, each of which is optionally substituted. The R^{1a} group may be introduced at any appropriate step during the synthesis of a compound of formula I.

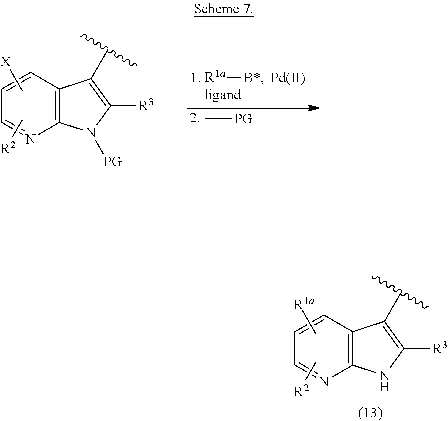

Compounds of formula I can be prepared according to the general procedures described in the Schemes and Examples herein, using appropriate materials. Representative compounds of the present invention are exemplified hereinbelow. The compounds exemplified are not, however, to be construed as limiting the scope of the invention in any manner. The examples further illustrate details for the preparation of the compounds of the present invention. Those skilled in the art will readily understand that known variations of protecting groups, of reagents, as well as of the conditions and processes of the following preparative procedures, can be used to prepare these compounds. It is also understood that whenever a chemical reagent is not commercially available, such a chemical reagent can be readily prepared by those skilled in the art by either following or adapting known methods described in the literature or described herein. Chiral analogs were prepared via incorporation of chiral reagents or obtained after chiral chromatography separation of a racemically generated analog. All temperatures are degrees Celsius unless otherwise noted. Mass spectra (MS) were measured either by electrospray ion-mass spectroscopy (ESMS) or by atmospheric pressure chemical ionization mass spectroscopy (APCI).

### Biological Assay

### JAK Biochemical HTRF Assay Protocol

The ability of compounds to inhibit the activity of JAK1, JAK2, JAK3, and TYK2 was measured using a recombinant purified GST-tagged catalytic domain for each enzyme (Invitrogen JAK1 #PV4774, JAK2 #PV4210, JAK3 #PV3855, TYK2 #PV4790) in an HTRF format biochemical assay. The reactions employed a common peptide substrate, LCB-EQEDEPEGDYFEWLW-NH₂. The basic assay protocol is as follows: First, 250 mL of diluted compounds in DMSO were dispensed into the wells of a dry 384-well Black plate (Greiner #781076) using a Labcyte Echo 555 acoustic dispenser. Subsequent reagent additions employed a Thermo Multidrop Combi nL. Next, 10 μL of 2× enzyme and 2× substrate in 1× assay buffer (Invitrogen kinase buffer #PV3189, 2 mM DTT, 0.05% BSA) were added to the wells and preincubated for 30 min at room temperature to allow compound binding to equilibrate. After equilibration, 10 μl, of 2×ATP in 1× assay buffer was added to initiate the kinase reaction and the plates were incubated at room temperature for 120 min. At the end of the incubation, 20 μL of 2× stop buffer containing streptavidin-Dylight (Thermo #21845) and Eu-tagged pY20 antibody (Perkin Elmer #AD0067) was added to quench the reaction. Plates were further incubated 60 min at room temperature with shaking and then read on a Perkin Elmer Envision (λₑₓ=337 nm, λₑₘ=665 and 615 nm, TRF delay time=20 μs). HTRF signal=10,000*665 nm reading/615 nm reading. After normalization to untreated controls, the percent inhibition of the HTRF signal at each compound concentration was calculated. The plot of percent inhibition vs. the log of compound concentration was fit with a 4-parameter dose response equation to calculate IC₅₀ values.

Final reaction conditions were:

| | [E] | [S] | [ATP] | [Eu-pY20] | [SA-Dylight] |
|---|---|---|---|---|---|
| Enzyme | (nM) | (μM) | (μM) | (nM) | (nM) |
| JAK1 | 1 | 2 | 30 | 9 | 313 |
| JAK2 | 0.015 | 2 | 5.3 | 9 | 313 |
| JAK3 | 0.015 | 2 | 1.7 | 9 | 313 |
| TYK2 | 2 | 4 | 1 | 18 | 625 |

Compound concentrations tested were 1482, 494, 173, 49, 19, 6.2, 2.1, 0.74, 0.24, 0.074, and 0.012 nM, with 1.2% residual DMSO. IC50 for representative compounds are provided as follows: ++++: ≦1 nM; +++: >1 nM ≦10 nM; ++: >10 nM ≦100 nM; +: >100 nM ≦1000 nM.

### Preparation of Intermediates

### Intermediate 1a. 5-Chloro-3-(4,4,5,5-tetramethyl-1,3,2-dioxaborolan-2-yl)-1-tosyl-1H-pyrrolo[2,3-b]pyridine

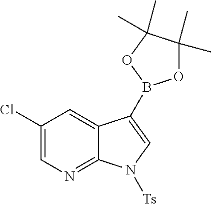 [SMILES: Cc1ccc(S(=O)(=O)n2cc(B3OC(C)(C)C(C)(C)O3)c3cc(Cl)cnc32)cc1]

### Step 1. 5-chloro-3-iodopyridin-2-amine

Into a 5000-mL 4-necked round-bottom flask was placed a solution of 5-chloro-pyridin-2-amine (520 g, 4.03 mol, 1.00 equiv) in ethanol (1560 mL) and water (360 mL). To this was added sulfuric acid (52 mL) dropwise with stirring. This was followed by addition of H5IO6 (190 g, 833.33 mmol, 0.20 equiv) in several batches at 30° C. To the mixture was added 12 (400 g, 1.57 mol, 0.40 equiv) in several batches. The resulting solution was stirred for 15 h at 80° C. in an oil bath. The resulting solution was diluted with 14000 mL of ice water. The pH value of the solution was adjusted to 9 with sodium hydroxide solution (5M). The solid was collected by filtration. Then it was dissolved in 20000 mL of EtOAc and washed with 1×1000 mL of Na₂S₂O₄ solution. The EtOAc solution was treated with sodium hydroxide solution (1M) till pH 9 was reached. The separated organic layer was washed with 2×1000 mL of brine, dried over anhydrous sodium sulfate and concentrated under vacuum. The residue was applied onto a silica gel column and eluted with ethyl acetate/petroleum ether (0˜0.03). This resulted in 580 g (56%) of 5-chloro-3-iodopyridin-2-amine as a brown solid.

### Step 2. 5-chloro-3-(2-(trimethylsilyl)ethynyl)pyridin-2-amine

Into a 3000-mL 4-necked round-bottom flask, purged and maintained with an inert atmosphere of nitrogen, was placed a solution of 5-chloro-3-iodopyridin-2-amine (580 g, 2.28 mol, 1.00 equiv) in tetrahydrofuran (1450 mL). This was followed by the addition of copper (I) iodide (4.6 g, 24.21 mmol, 0.01 equiv), PdCl₂(PPh₃)₂ (16 g, 22.79 mmol, 0.01 equiv) and triethylamine (960 mL). To the reaction mixture was added ethynyltrimethylsilane (285.6 g, 2.91 mol, 1.30 equiv) dropwise with stirring at 20-40° C. over 40 min. After stirred for an additional 60 min at room temperature, the reaction mixture was cooled to 0° C. with an ice water bath and diluted with 3000 mL of ether. The isolated solid was filtered out and washed with ether (3×1000 ml). The filtrate was dried over Na₂SO₄ and concentrated under vacuum. This resulted in 446.3 g (87%) of 5-chloro-3-(2-(trimethylsilyl)ethynyl)pyridin-2-amine as a yellow solid.

### Step 3. 5-chloro-1H-pyrrolo[2,3-b]pyridine

Into a 10000-mL 4-necked round-bottom flask, purged and maintained with an inert atmosphere of nitrogen, was placed a solution of potassium t-butoxide (446.3 g, 3.98 mol, 2.00 equiv) in NMP (1300 mL). This was followed by the addition of a solution of 5-chloro-3-(2-(trimethylsilyl)ethynyl)pyridin-2-amine (446.3 g, 1.98 mol, 1.00 equiv) in NMP (2500 mL) dropwise with stirring at 80° C. over 3 hours. After stirred for 60 min at 80° C. in an oil bath, the reaction mixture was cooled to 40° C. and diluted with 7500 mL of brine. The resulting solution was extracted with 5×3000 mL of ether. The organic layers were combined, washed with 2×3000 mL of brine, dried over anhydrous sodium sulfate and concentrated under vacuum. The residue was applied onto a silica gel column and eluted with PE to EtOAc:PE (1˜30). This resulted in 120 g (40%) of 5-chloro-1H-pyrrolo[2,3-b]pyridine as a yellow solid.

### Step 4. 3-bromo-5-chloro-1H-pyrrolo[2,3-b]pyridine

Into a 3000-mL 3-necked round-bottom flask was placed a solution of 5-chloro-1H-pyrrolo[2,3-b]pyridine (120 g, 784.31 mmol, 1.00 equiv) in AcOH (1200 mL). This was followed by the addition of a solution of Br₂ (139 g, 868.75 mmol, 1.10 equiv) in AcOH (600 mL) dropwise with stirring at 90° C. over 80 min. The resulting solution was stirred for 1.5 h at 90° C. in an oil bath. Then it was cooled and concentrated under vacuum. The residue was adjusted to pH 10 with sodium hydroxide solution (1M). The solid was collected by filtration, then washed with 3×500 ml of water and dried in an oven under reduced pressure. This resulted in 87.2 g (48%) of 3-bromo-5-chloro-1H-pyrrolo[2,3-b]pyridine as a yellow solid.

### Step 5. 3-bromo-5-chloro-1-tosyl-1H-pyrrolo[2,3-b]pyridine

Into a 1000-mL 4-necked round-bottom flask, purged and maintained with an inert atmosphere of nitrogen, was placed a solution of 3-bromo-5-chloro-1H-pyrrolo[2,3-b]-pyridine (87.5 g, 378.79 mmol, 1.00 equiv) in N,N-dimethylformamide (600 mL). This was followed by the addition of sodium hydride (18.3 g, 457.50 mmol, 1.20 equiv, 60%) in several batches at 0° C. over 20 min. The reaction mixture was stirred for 30 min at 0° C., then added 4-methylbenzene-1-sulfonyl chloride (72.5 g, 379.58 mmol, 1.05 equiv) in several batches at 0° C. over 30 min. The resulting solution was stirred for 1 h at 25° C., then quenched by the addition of 1200 mL of ice water. The solid was collected by filtration, then washed with 3×800 ml of water and dried in an oven under reduced pressure. This resulted in 122.2 g (84%) of 3-bromo-5-chloro-1-tosyl-1H-pyrrolo[2,3-b]pyridine as a yellow solid.

### Step 6. 5-chloro-3-(4,4,5,5-tetramethyl-1,3,2-dioxaborolan-2-yl)-1-tosyl-1H-pyrrolo[2,3-b]-pyridine

Into a 3000-mL 4-necked round-bottom flask, purged and maintained with an inert atmosphere of nitrogen, were placed a solution of 3-bromo-5-chloro-1-tosyl-1H-pyrrolo[2,3-b]pyridine (101.5 g, 262.95 mmol, 1.00 equiv) in ethylene glycol dimethyl ether (1400 mL), 4,4,5,5-tetramethyl-2-(4,4,5,5-tetramethyl-1,3,2-dioxaborolan-2-yl)-1,3,2-dioxa-borolane (101.5 g, 399.61 mmol, 1.50 equiv), PdCl₂(dppf)₂ (22 g, 26.41 mmol, 0.10 equiv) and AcOK (78 g, 795.92 mmol, 3.00 equiv). The resulting mixture was stirred for 6 h at 86° C. in an oil bath. The reaction mixture was cooled and diluted with 2800 mL of EtOAc and 1000 mL of brine. The solid was filtered out. The filtrate was washed with 3×1000 ml of brine. The organic layer was dried over anhydrous sodium sulfate and concentrated under vacuum. The crude product was re-crystallized from EtOAc/PE in the ratio of 1:1. to provide the title compound as a gray solid. LC-MS (ES, m/z): 433 [M+H]+. 1H-NMR (400 Hz, CDCl₃, ppm): 8.361 (1H, d, J=1.5 Hz), 8.176 (2H, d, J=1.5 Hz), 8.097 (2H, m), 7.307 (2H, m), 2.396 (3H, s), 1.376 (12H, s).

### Intermediate 1b. Potassium [5-chloro-1-(4-methylphenylsulfonyl)-1H-pyrrolo[2,3-b]pyridin-3-yl](trifluoro)borate

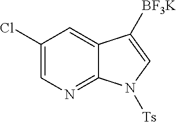

Intermediate 1a (19.6 g, 46.8 mmol) was dissolved in acetone (181 ml) and water (91 ml). Potassium hydrogen fluoride (21.94 g, 281 mmol) was added and the mixture stirred at RT overnight. Upon disappearance of the boronic ester starting material, the reaction mixture was concentrated to dryness and triturated w/hot acetone (55° C.). The solids were filtered and the filtrate concentrated to a minimal volume. While hot, ether was added. The product oiled out and was stored in a freezer overnight. The resulting solid was collected. The material was used without further purification.

### Intermediate 2. (2E)-3-(6-chloropyrazin-2-yl)-2-methyl-N-(2,2,2-trifluoroethyl)prop-2-enamide

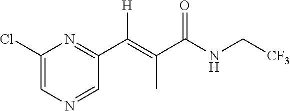 [SMILES: C/C(=C\c1cncc(Cl)n1)C(=O)NCC(F)(F)F]

### Step 1. ethyl (2E)-3-(6-chloropyrazin-2-yl)-2-methyl-2-enoate

To a solution of 6-chloropyrazine-2-carbaldehyde (150 mg, 1.052 mmol) in benzene (7.3 ml) was added ethyl 2-(triphenyl-λ⁵-phosphanylidene)propanoate (572 mg, 1.578 mmol), with stirring. The mixture was then heated to 85° C. in a sealed vial and aged for 16 hours. The reaction mixture was then concentrated and purified directly on NPLC, eluting with ethyl acetate/isohexanes (0-80%) to afford ethyl (2E)-3-(6-chloropyrazin-2-yl)-2-methylprop-2-enoate as colorless crystals.

### Step 2. (2E)-3-(6-chloropyrazin-2-yl)-2-methyl-N-(2,2,2-trifluoroethyl)prop-2-enamide

To a solution of the product of Step 1 (239 mg, 1.054 mmol) in methanol (5 mL), was added aq. 1N sodium hydroxide (2.20 mL, 2.20 mmol). The white suspension was stirred at 23° C. for 1 hr. The solution was neutralized with 1M HCl and the mixture was concentrated in vacuo to afford the crude carboxylic acid, which was then re-dissolved in DMF (2.2 ml). To this solution were added Py-BOP (524 mg, 1.00 mmol), 2,2,2-trifluoroethanamine hydrochloride (273 mg, 2.014 mmol) and Hunig's base (0.880 ml, 5.0 mmol). The resulting mixture was stirred at 23° C. for 5 min, and subsequently quenched with 5 mL water. The mixture was then concentrated in vacuo and purified directly on NPLC, eluting with ethyl acetate/isohexanes (50-100%) to afford the title compound as a colorless solid.

### Intermediate 3. 4-(6-bromopyrazin-2-yl)-N-(2,2,2-trifluoroethyl)benzamide

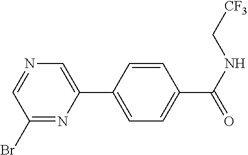 [SMILES: O=C(NCC(F)(F)F)c1ccc(-c2cncc(Br)n2)cc1]

2,6-Dibromopyrazine (72 mg, 0.30 mmol), PdCl₂(dppf) (30 mg, 0.40 mmol), sodium carbonate (34 mg, 0.32 mmol) and {4-[(2,2,2-trifluoroethyl)carbamoyl]phenyl}boronic acid (50 mg, 0.20 mmol) were dissolved in 1,4-dioxane (1 mL) and water (0.2 mL) under nitrogen in a 4-mL vial and heated to 80° C. for 1.5 h. The residue was purified by preparative HPLC reverse phase (C-18), loading in MeOH with TFA, eluting with acetonitrile/water+0.1% TFA (eluting with 10->100% MeCN), to give the title compound TFA salt as a yellow solid.

### Intermediate 4. methyl 3-(6-chloropyrazin-2-yl)-2-methylpropanoate

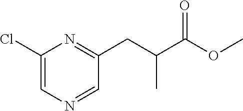

To a flask charged with Ar was added 2,6-dichloropyrazine (0.5 g, 3.36 mmol), Pd(Ph₃P)₄ (0.078 g, 0.067 mmol). The flask was again purged with Argon for an additional 5 min. THF (2.80 ml) was introduced to the flask, and the resulting mixture was degassed for 5 min. The flask was placed in a RT water bath and a brown solution of (R)-(+)-3-methoxy-2-methyl-3-oxopropylzinc bromide solution (7.38 ml, 3.69 mmol) was introduced drop wise to reaction mixture. The resulting solution was degassed for another 5 min and then heated to 70° C. and allowed to stir. Removed from heat after 4 hours and allowed to cool to RT overnight. The reaction mixture was diluted with water and Et₂O. The layers were separated and the organic layer was washed with sat. sodium bicarbonate and brine. The combined aqueous layers were back extracted once with Et₂O. Combined organic layers were dried with sodium sulfate, filtered and concentrated. The residue was purified by flash column chromatography on silica gel (EtOAc/hexanes gradient, 5-50%) to give colorless oil.

### General Scheme for Example 1

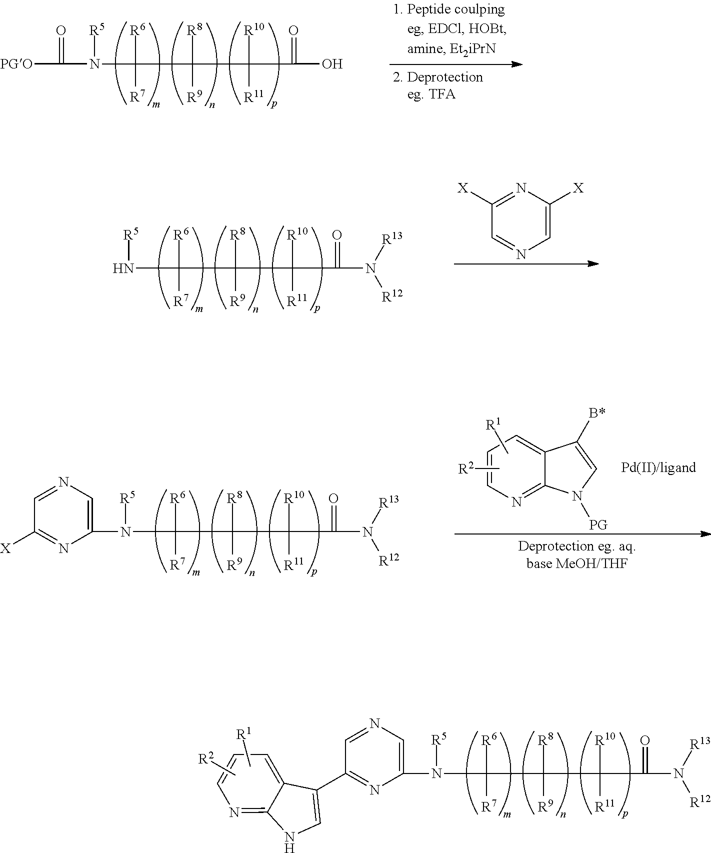

### Example 1

### N²-[6-(5-Chloro-1H-pyrrolo[2,3-b]pyridin-3-yl)pyrazin-2-yl]-N-(2,2,2-trifluoroethyl)-D-alaninamide

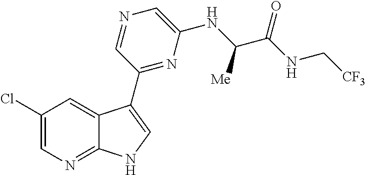 [SMILES: C[C@@H](Nc1cncc(-c2c[nH]c3ncc(Cl)cc23)n1)C(=O)NCC(F)(F)F]

### Step 1. N²-(tert-butoxycarbonyl)-N-(2,2,2-trifluoroethyl)-D-alaninamide

To a stirred solution of N-(tert-butoxycarbonyl)-D-alanine (5 g, 26.4 mmol), EDC (6.08 g, 31.7 mmol), HOBT (5.26 g, 34.4 mmol), Hünig's base (13.85 ml, 79 mmol) in Dichloromethane (132 ml) was added 2,2,2-trifluoroethanamine hydrochloride (3.94 g, 29.1 mmol) and the reaction was stirred for 16 h. The mixture was then diluted with dichloromethane and washed with water and brine. The organic layer was dried over magnesium sulfate. The solvent was evaporated and the resulting colorless oil was diluted with benzene and lyophilized under high vacuum to afford title compound as a colorless solid. The material was used directly in the subsequent step without purification.

### Step 2. N-(2,2,2-trifluoroethyl)-D-alaninamide.TFA

The product of Step 1 (1.5 g, 5.55 mmol) was dissolved in dichloromethane (15 mL) and TFA (10 ml, 130 mmol) was added. The yellowish solution was stirred at room temperature for 45 min. Solvent evaporation gave crude title compound as the TFA salt, which was used without further purification in the subsequent step.

### Step 3. N²-(6-chloropyrazin-2-yl)-N-(2,2,2-trifluoroethyl)-D-alaninamide.TFA

A round vial with a sealed septum screw-cap was charged with 2,6-dichloro-pyrazine (276 mg, 1.851 mmol) and the crude amine TFA salt obtained in Step 2 (526 mg, 1.851 mmol). Anhydrous DMSO (9.0 mL) and triethylamine (780 μl, 5.60 mmol) were added and the reaction mixture heated to 100° C. for 16 h. After cooling to room temperature the reaction mixture was directly purified by preparative HPLC Reverse phase (C-18), loading in DMSO with TFA, eluting with Acetonitrile/Water+0.1% TFA (eluting with 10->70% MeCN, 100% wash). The major peak was collected and concentrated in vacuo to afford the TFA salt of the title compound as light brown paste of sufficient purity for the subsequent step.

### Step 4. N²-(6-{5-chloro-1-[(4-methylphenyl)sulfonyl]-1H-pyrrolo[2,3-b]pyridin-3-yl}pyrazin-2-yl)-N-(2,2,2-trifluoroethyl)-D-alaninamide.TFA

The product of Step 3 (100 mg, 0.252 mmol), PdCl₂(dppf) (37 mg, 0.051 mmol), potassium {5-chloro-1-[(4-methylphenyl)sulfonyl]-1H-pyrrolo[2,3-b]pyridin-3-yl}(trifluoro)-borate (112 mg, 0.252 mmol) and triethylamine (150 μl, 1.076 mmol) were suspended in anhydrous n-PrOH (1260 μl) under nitrogen and heated to 100° C. for 16 h. The reaction mixture was filtered through celite with methanol. The solvents were evaporated. The residue was purified by preparative HPLC Reverse phase (C-18), loading in MeOH with TFA, eluting with Acetonitrile/Water+0.1% TFA (eluting with 20->80% MeCN, 100% wash). The third peak was collected and concentrated in vacuo to give the crude title compound as a dark solid, which was directly used in the subsequent step.

### Step 5. N²-[6-(5-chloro-1H-pyrrolo[2,3-b]pyridin-3-yl)pyrazin-2-yl]-N-(2,2,2-trifluoroethyl)-D-alaninamide.TFA

The product of Step 4 (136 mg, 0.252 mmol) was dissolved in methanol/THF (1:1; 4 mL) and aqueous sodium hydroxide solution (1M, 2.8 mL) was added. The reaction mixture was stirred for 1 hour, then purified by preparative HPLC Reverse phase (C-18), loading in MeOH with TFA, eluting with acetonitrile/water+0.1% TFA (eluting with 0->70% MeCN), to give the TFA salt of the title compound as a yellow solid. APCI: [M+H]⁺ m/z 399.1. ¹H NMR (500 MHz, dmso) δ 12.32 (s, 1H), 8.66 (s, 1H), 8.63 (s, 1H), 8.31 (d, J=2.8, 1H), 8.30 (s, 1H), 8.25 (d, J=2.4, 1H), 7.80 (s, 1H), 7.30 (s, 1H), 4.48-4.37 (m, 1H), 4.01-3.89 (m, 1H), 3.89-3.78 (m, 1H), 1.41 (d, J=7.1, 3H). rhJAK3 IC₅₀: ++++

The following examples were prepared in an analogous manner of that described in GENERAL SCHEME FOR EXAMPLE 1 using materials that are commercially available or known, or that can be prepared using procedures known in the art or by generally following procedures described herein for various intermediates.

| | | MS | rhJAK3 |
|---|---|---|---|
| Example | Structure/Chemical Name | [M + H]+ | IC₅₀ |
| 1-1 | 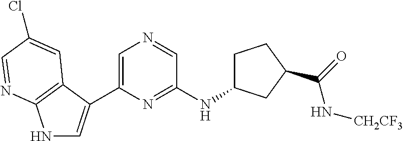 (1R,3R)-3-{[6-(5-chloro-1H-pyrrolo[2,3-b]pyridin-3-yl)- pyrazin-2-yl]amino}-N-(2,2,2-trifluoroethyl)cyclopentane- carboxamide | 439.1 | +++ |
| 1-2 | 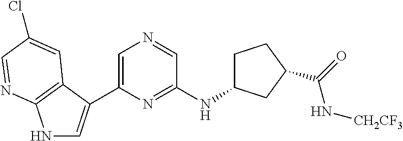 (1S,3R)-3-{[6-(5-chloro-1H-pyrrolo[2,3-b]pyridin-3-yl)- pyrazin-2-yl]amino}-N-(2,2,2-trifluoroethyl)cyclopentane- carboxamide | 439.1 | +++ |
| 1-3 | 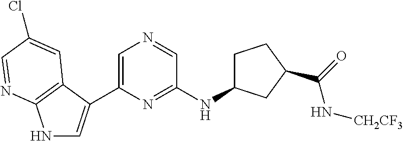 (1R,3S)-3-{(6-(5-chloro-1H-pyrrolo[2,3-b]pyridin-3-yl)- pyrazin-2-yl]amino}-N-(2,2,2-trifluoroethyl)cyclopentane- carboxamide | 439.1 | +++ |
| 1-4 | 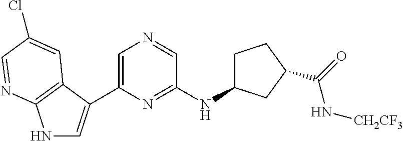 (1S,3S)-3-{[6-(5-chloro-1H-pyrrolo[2,3-b]pyridin-3-yl)- pyrazin-2-yl]amino}-N-(2,2,2-trifluoroethyl)cyclopentane- carboxamide | 439.1 | ++ |
| 1-5 | 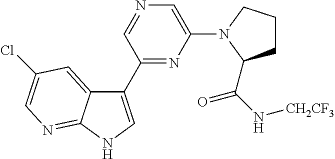 1-[6-(5-chloro-1H-pyrrolo[2,3-b]pyridin-3-yl)-pyrazin-2-yl]- N-(2,2,2-trifluoroethyl)-L-prolinamide | 425.1 | +++ |
| 1-6 | 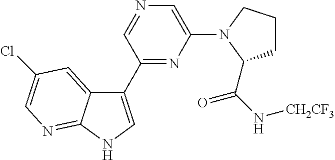 1-[6-(5-chloro-1H-pyrrolo[2,3-b]pyridin-3-yl)pyrazin-2-yl]- N-(2,2,2-trifluoroethyl)-D-prolinamide | 425.1 | ++++ |
| 1-7 | 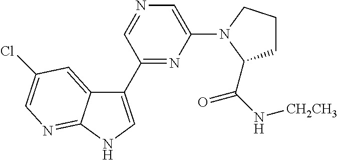 [SMILES: CCNC(=O)[C@H]1CCCN1c1cncc(-c2c[nH]c3ncc(Cl)cc23)n1] 1-[6-(5-chloro-1H-pyrrolo[2,3-b]pyridin-3-yl)pyrazin-2-yl]- N-ethyl-D-prolinamide | 371.1 | +++ |
| 1-8 | 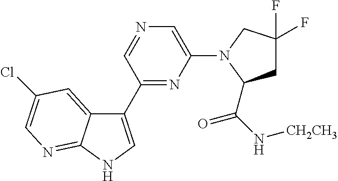 [SMILES: CCNC(=O)[C@@H]1CC(F)(F)CN1c1cncc(-c2c[nH]c3ncc(Cl)cc23)n1] 1-[6-(5-chloro-1H-pyrrolo[2,3-b]pyridin-3-yl)pyrazin-2-yl]- 4,4-difluoro-N-(2,2,2-trifluoroethyl)-L-prolinamide | 461.1 | +++ |
| 1-9 | 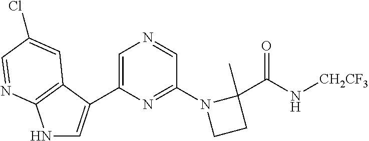 1-[6-(5-chloro-1H-pyrrolo[2,3-b]pyridin-3-yl)pyrazin-2-yl]- 2-methyl-N-(2,2,2-trifluoroethyl)azetidine-2-carboxamide | 425.1 | ++++ |
| 1-10 | 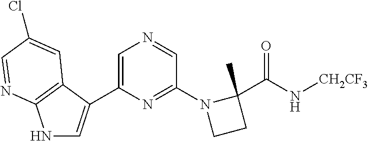 (2S)-1-[6-(5-chloro-1H-pyrrolo[2,3-b]pyridin-3-yl)pyrazin-2- yl]-2-methyl-N-(2,2,2-trifluoroethyl)azetidine-2-carboxamide | 425.1 | +++ |
| 1-11 | 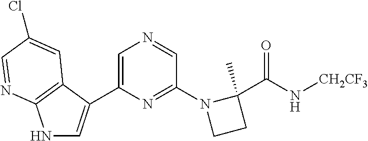 (2R)-1-[6-(5-chloro-1H-pyrrolo[2,3-b]pyridin-3-yl)pyrazin-2- yl]-2-methyl-N-(2,2,2-trifluoroethyl)azetidine-2-carboxamide | 425.1 | ++++ |
| 1-12 | 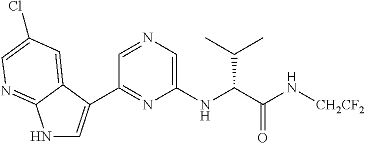 N²-[6-(5-chloro-1H-pyrrolo[2,3-b]pyridin-3-yl)pyrazin-2-yl]- N-(2,2,2-trifluoroethyl)-D-valinamide | 426.2 | ++++ |
| 1-13 | 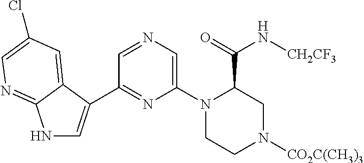 tert-butyl (3R)-4-[6-(5-chloro-1H-pyrrolo[2,3-b]pyridin-3- yl)pyrazin-2-yl]-3-[(2,2,2-trifluoroethyl)carbamoyl]- piperazine-1-carboxylate | 539.3 | +++ |
| 1-14 | 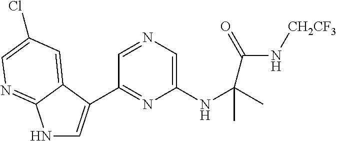 N²-[6-(5-chloro-1H-pyrrolo[2,3-b]pyridin-3-yl)pyrazin-2-yl]- 2-methyl-N-(2,2,2-trifluoroethyl)alaninamide | 413.1 | ++++ |
| 1-15 | 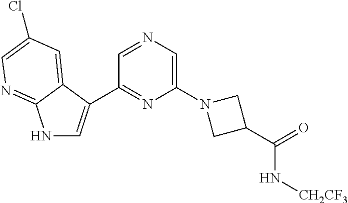 1-[6-(5-chloro-1H-pyrrolo[2,3-b]pyridin-3-yl)pyrazin-2-yl]- N-(2,2,2-trifluoroethyl)azetidine-3-carboxamide | 411.0 | ++ |
| 1-16 | 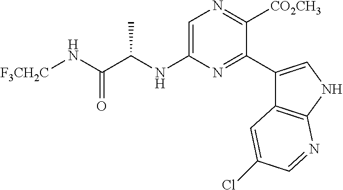 methyl 3-(5-chloro-1H-pyrrolo[2,3-b]pyridin-3-yl)-5-({(1S)- 1-methyl-2-oxo-2-[(2,2,2-trifluoroethyl)amino]ethyl}amino)- pyrazine-2-carboxylate | 457.2 | ++ |

### General Scheme for Example 2

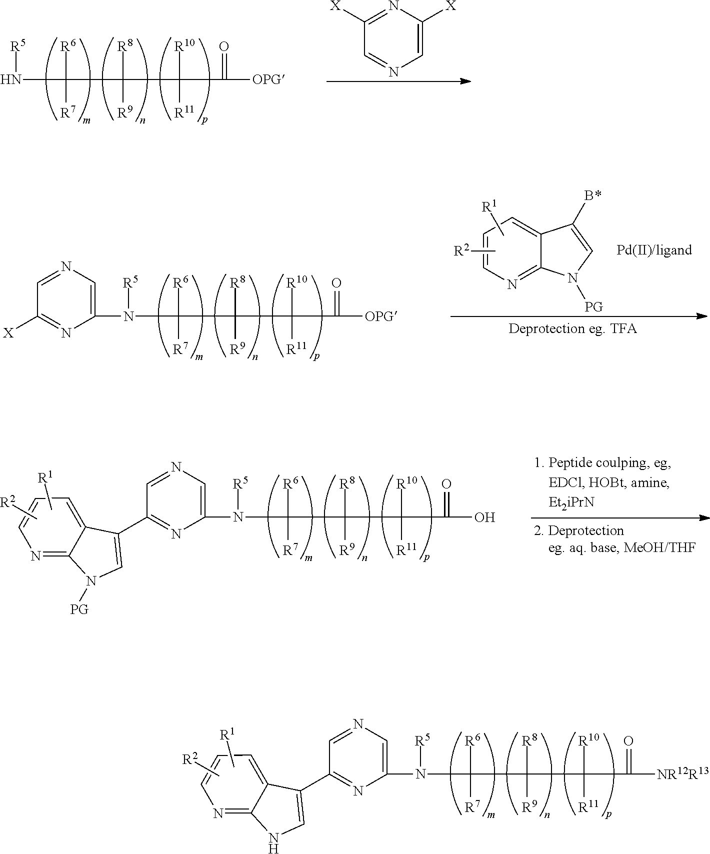

### Example 2

### N²-[6-(5-Chloro-1H-pyrrolo[2,3-b]pyridin-3-yl)pyrazin-2-yl]-N-(2,2,2-trifluoroethyl)-L-alaninamide

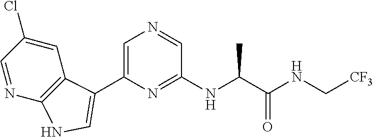 [SMILES: C[C@H](Nc1cncc(-c2c[nH]c3ncc(Cl)cc23)n1)C(=O)NCC(F)(F)F]

### Step 1. tert-butyl N-(6-chloropyrazin-2-yl)-L-alaninate

2,6-Dichloropyrazine (2 g, 13.42 mmol), tert-butyl L-alaninate (2.463 g, 13.56 mmol) and DIEA (7 ml, 40.1 mmol) were added to a flask and flushed with N2. 2-Propanol (26 ml) was added and the reaction was stirred at 80° C. for 16 h. The product was purified on silica gel to afford the title compound. MS APCI: [M+H]⁺ m/z 258.1.

### Step 2. tert-butyl N-{6-[5-chloro-1-(phenylsulfonyl)-1H-pyrrolo[2,3-b]pyridin-3-yl]pyrazin-2-yl}-L-alaninate

The product of Step 1 (240.6 mg, 0.934 mmo), potassium [5-chloro-1-(phenyl-sulfonyl)-1H-pyrrolo[2,3-b]pyridin-3-yl](trifluoro)borate(1-) (680.5 mg, 1.707 mmol), PdCl₂(dppf) (167.1 mg, 0.205 mmol) and triethylamine (0.390 mL, 2.80 mmol) were suspended in nPrOH (4 mL, Density: 0.79 g/ml) and heated to 100° C. for 1 h. The mixture was diluted with DCM and washed with water, brine, dried and concentrated to afford residue, which was purified on silica gel (EtOAc/hexane=1:1) to afford the title compound. MS APCI: [M+H]⁺ m/z 514.1.

### Step 3. N-{6-[5-chloro-1-(phenylsulfonyl)-1H-pyrrolo[2,3-b]pyridin-3-yl]pyrazin-2-yl}-L-alanine

The product of Step 2 (234.4 mg, 0.456 mmol) was dissolved into DCM (1 ml). To this solution, was added TFA (1 ml, 12.98 mmol). The mixture was stirred at rt for overnight. The solvent was evaporated to afford the title compound. MS APCI: [M+H]⁺ m/z 458.1.

### Step 4. N²-{6-[5-chloro-1-(phenylsulfonyl)-1H-pyrrolo[2,3-b]pyridin-3-yl]pyrazin-2-yl}-N-(2,2,2-trifluoroethyl)-L-alaninamide

To a flask, were added the product of Step 3 (194.7 mg, 0.340 mmol), 2,2,2-trifluoroethanamine (90.6 mg, 0.669 mmol), PyBOP (234.8 mg, 0.451 mmol), DIEA (0.3 ml, 1.718 mmol) and DMF (1.5 ml). The mixture was stirred at rt for 30 min. and purified on silica gel (DCM/MeOH=94/6) to afford the title compound. MS APCI: [M+H]⁺ m/z 539.0.

### Step 5. N²-[6-(5-chloro-1H-pyrrolo[2,3-b]pyridin-2-yl]-N-(2,2,2-trifluoroethyl)-L-alaninamide

To a flask, were added the product of Step 4 (152.4 mg, 0.283 mmol), K₂CO₃ (117 mg, 0.848 mmol) and MeOH (3 ml). The mixture was heated at 50° C. for 1 h., filtered and purified on RP HPLC to afford the title product. MS APCI: [M+H]⁺ m/z 399.1. ¹H NMR (500 MHz, DMSO) δ 12.32 (s, 1H), 8.66 (t, 1H), 8.62 (d, 1H), 8.31 (d, 1H), 8.30 (s, 1H), 8.24 (d, 1H), 7.80 (s, 1H), 7.34 (s, 1H), 4.42 (q, 1H), 3.55-4.00 (m, 2H), 1.41 (d, 3H). rhJAK3 IC₅₀: ++++

The following examples were prepared in an analogous manner of that described in GENERAL SCHEME FOR EXAMPLE 2 using materials that are commercially available or known, or that can be prepared using procedures known in the art or by generally following procedures described herein for various intermediates.

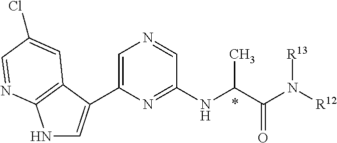

| | | | MS | rhJAK3 |
|---|---|---|---|---|
| Example | NR¹²R¹³, (*) | Chemical Name | [M + H]+ | IC₅₀ |
| 2-1 | NHCH₂CN, (S) | N²-[6-(5-chloro-1H-pyrrolo[2,3-b]- pyridin-3-yl)pyrazin-2-yl]-N- (cyanomethyl)-L-alaninamide | 356.1 | ++ |
| 2-2 | 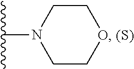 | 6-(5-chloro-1H-pyrrolo[2,3-b]- pyridin-3-yl)-N-[(1S)-1-methyl-2- morpholin-4-yl-2-oxoethyl]pyrazin- 2-amine | 387.1 | + |
| 2-3 | 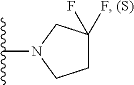 | 6-(5-chloro-1H-pyrrolo[2,3-b]- pyridin-3-yl)-N-[(1S)-2-(3,3- difluoropyrrolidin-1-yl)-1-methyl- 2-oxoethyl]pyrazin-2-amine | 407.1 | ++ |
| 2-4 | 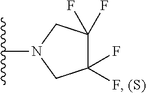 | 6-(5-chloro-1H-pyrrolo[2,3-b]- pyridin-3-yl)-N-[(1S)-1-methyl-2- oxo-2-(3,3,4,4-tetrafluoro- pyrrolidin-1-yl)ethyl]pyrazin-2- amine | 443.1 | + |
| 2-5 | 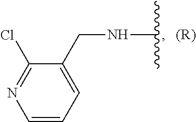 | N-[(2-chloropyridin-3-yl)methyl]- N²-[6-(5-chloro-1H-pyrrolo[2,3-b]- pyridin-3-yl)pyrazin-2-yl]-D- alaninamide | 442.1 | + |
| 2-6 | 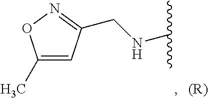 [SMILES: *NCc1cc(C)on1 ; "(R)"] | N²-[6-(5-chloro-1H-pyrrolo[2,3-b]- pyridin-3-yl)pyrazin-2-yl]-N-[(5 - methylisoxazol-3-yl)methyl]-D- alaninamide | 412.1 | + |
| 2-7 | 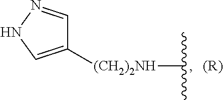 | N²-[6-(5-chloro-1H-pyrrolo[2,3-b]- pyridin-3-yl)pyrazin-2-yl]-N-[2- (1H-pyrazol-4-yl)ethyl]-D- alaninamide | 411.1 | + |
| 2-8 | 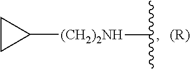 | N²-[6-(5-chloro-1H-pyrrolo[2,3-b]- pyridin-3-yl)pyrazin-2-yl]-N-(2- cyclopropylethyl)-D-alaninamide | 385.1 | ++ |
| 2-9 | 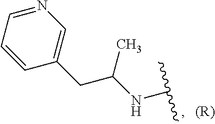 | N²-[6-(5-chloro-1H-pyrrolo[2,3-b]- pyridin-3-yl)pyrazin-2-yl]-N-(l - methyl-2-pyridin-3-ylethyl)-D- alaninamide | 218.8 | + |
| 2-10 | 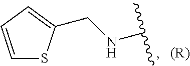 | N²-[6-(5-chloro-1H-pyrrolo[2,3-b]- pyridin-3-yl)pyrazin-2-yl]-N- (thiophen-2-ylmethyl)-D- alaninamide | 413.1 | + |
| 2-11 | 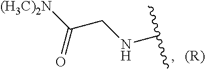 | N-[6-(5-chloro-1H-pyrrolo[2,3-b]- pyridin-3-yl)pyrazin-2-yl]-D-alanyl- N,N-dimethylglycinamide | 402.1 | + |
| 2-12 | 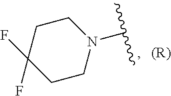 | 6-(5-chloro-1H-pyrrolo[2,3-b]- pyridin-3-yl)-N-[(1R)-2-(4,4- difluoropiperidin-1-yl)-1-methyl-2- oxoethyl]pyrazin-2-amine | 421.1 | + |
| 2-13 | 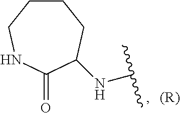 | N²-[6-(5-chloro-1H-pyrrolo[2,3-b]- pyridin-3-yl)pyrazin-2-yl]-N-(2- oxoazepan-3-yl)-D-alaninamide | 428.2 | + |
| 2-14 | 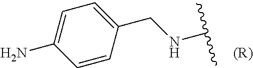 | N-(4-aminobenzyl)-N²-[6-(5- chloro-1H-pyrrolo[2,3-b]pyridin-3- yl)pyrazin-2-yl]-D-alaninamide | 422.1 | + |
| 2-15 | 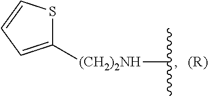 | N²-[6-(5-chloro-1H-pyrrolo[2,3-b]- pyridin-3-yl)pyrazin-2-yl]-N-(2- thiophen-2-ylethyl)-D-alaninamide | 427.1 | + |
| 2-16 | 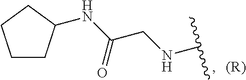 | N-[6-(5-chloro-1H-pyrrolo[2,3-b]- pyridin-3-yl)pyrazin-2-yl]-D-alanyl- N-cyclopentylglycinamide | 442.2 | + |
| 2-17 | 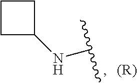 | N²-[6-(5-chloro-1H-pyrrolo[2,3-b]- pyridin-3-yl)pyrazin-2-yl]-N-cyclo- butyl-D-alaninamide | 371.1 | ++ |
| 2-18 | 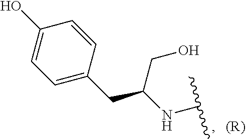 | N²-[6-(5-chloro-1H-pyrrolo[2,3-b]- pyridin-3-yl)pyrazin-2-yl]-N-[(1S)- 2-hydroxy-1-(4-hydroxybenzyl)- ethyl]-D-alaninamide | 467.2 | + |
| 2-19 | 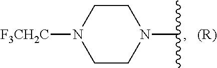 | 6-(5-chloro-1H-pyrrolo[2,3-b]- pyridin-3-yl)-N-{(1R)-1-methyl-2- oxo-2-[4-(2,2,2-trifluoroethyl)- piperazin-1-yl]ethyl}pyrazin-2- amine | 468.1 | + |
| 2-20 | 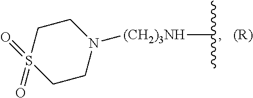 | N²-[6-(5-chloro-1H-pyrrolo[2,3-b]- pyridin-3-yl)pyrazin-2-yl]-N-[3- (1,1-dioxidothiomorpholin-4-yl)- propyl]-D-alaninamide | 492.1 | + |
| 2-21 | 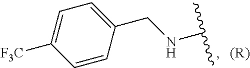 | N²-[6-(5-chloro-1H-pyrrolo[2,3-b]- pyridin-3-yl)pyrazin-2-yl]-N-[4- (trifluoromethyl)benzyl]-D- alaninamide | 475.1 | + |
| 2-22 | NH(CH₂)₂CF₃, (S) | N²-[6-(5-chloro-1H-pyrrolo[2,3-b]- pyridin-3-yl)pyrazin-2-yl]-N-(3,3,3- trifluoropropyl)-L-alaninamide | 413.1 | ++ |
| 2-23 | NHCH₂CH(CH₃)₂, (S) | N²-[6-(5-chloro-1H-pyrrolo[2,3-b]- pyridin-3-yl)pyrazin-2-yl]-N-(2- methylpropyl)-L-alaninamide | 373.2 | ++ |
| 2-24 | NHCH₂C(CH₃)₃, (S) | N²-[6-(5-chloro-1H-pyrrolo[2,3-b]- pyridin-3-yl)pyrazin-2-yl]-N-(2,2- dimethylpropyl)-L-alaninamide | 387.2 | + |

| | | | |
|---|---|---|---|
| 2-25 | 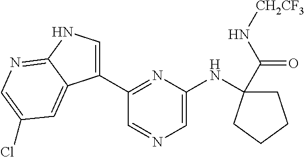 1-{[6-(5-chloro-1H-pyrrolo[2,3-b]pyridin-3-yl)pyrazin-2- yl]amino}-N-(2,2,2-trifluoroethyl)cyclopentanecarboxamide | 438.2 | ++++ |
| 2-26 | 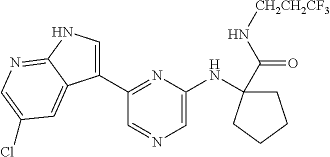 1-{[6-(5-chloro-1H-pyrrolo[2,3-b]pyridin-3-yl)pyrazin-2- yl]amino}-N-(3,3,3-trifluoropropyl)cyclopentanecarboxamide | 452.2 | + |

### Example 3

### (2S,3S)-1-[6-(5-Chloro-1H-pyrrolo[2,3-b]pyridin-3-yl)pyrazin-2-yl]-3-hydroxy-N-(2,2,2-trifluoroethyl)azetidine-2-carboxamide

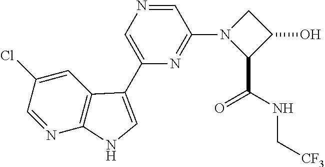 [SMILES: O=C(NCC(F)(F)F)[C@@H]1[C@@H](O)CN1c1cncc(-c2c[nH]c3ncc(Cl)cc23)n1]

### Step 1. methyl (2S,3S)-3-{[tert-butyl(dimethyl)silyl]oxy}azetidine-2-carboxylate

To the solution of 1-tert-butyl 2-methyl (2S,3S)-3-{[tert-butyl(dimethyl)silyl]-oxy}azetidine-1,2-dicarboxylate (488.2 mg, 1.413 mmol) in DCM (1 ml), was added TFA (1 ml, 12.98 mmol). The mixture was stirred at rt for 2 h., and the solvent was evaporated and the residue was used in next step directly. MS APCI: [M+H]⁺ m/z 246.1.

### Step 2. methyl (2S,3S)-3-{[tert-butyl(dimethyl)silyl]oxy}-1-(6-chloropyrazin-2-yl)azetidine-2-carboxylate

To a vial were added the product of Step 1 (347 mg, 1.414 mmol), 2,6-dichloro-pyrazine (254.7 mg, 1.710 mmol), DIEA (0.8 ml, 4.58 mmol), DABCO (31 mg, 0.276 mmol) and 2-propanol (5 ml). The mixture was heated at 60° C. for 16 h. Additional DABCO (140 mg) and DIEA (0.8 ml) were added, and the mixture was heated at 80° C. for 5 h. The mixture was concentrated and purified on sillica gel (EtOAc/hexane=1/9) to afford the title compound. MS APCI: [M+H]⁺ m/z 358.1.

### Step 3. (2S,3S)-3-{[tert-butyl(dimethyl)silyl]oxy}-1-(6-chloropyrazin-2-yl)azetidine-2-carboxylic acid

To the solution of the product of Step 2 (69.5 mg, 0.194 mmol) in THF (1 ml), were added 1 M NaOH (0.6 ml, 0.600 mmol) and MeOH (0.5 ml). The mixture was stirred at rt for 30 min., evaporated and carried on to the next step directly. MS APCI: [M+H]⁺ m/z 344.1.

### Step 4. (2S,3S)-3-{[tert-butyl(dimethyl)silyl]oxy}-1-(6-chloropyrazin-2-yl)-N-(2,2,2-trifluoro-ethyl)azetidine-2-carboxamide

To a vial were added the product of Step 3 (66 mg, 0.192 mmol), 2,2,2-trifluoro-ethylamine (77.8 mg, 0.574 mmol), PyBOP (110 mg, 0.211 mmol) and DMF (2 ml). To this mixture was added DIEA (0.15 ml, 0.859 mmol). The mixture was stirred at rt for 8 h., diluted with EtOAc and washed with water. The organic phase was washed with brine, dried and concentrated to afford residue, which was purified on silica gel (EtOAc/hexane=3/7) to afford the title compound. MS APCI: [M+H]⁺ m/z 425.2.

### Step 5. 2S,3S-3-{[tert-butyl(dimethyl)silyl]oxy}-1-(6-{5-chloro-1-[4-methylphenyl)sulfonyl]-1H-pyrrolo[2,3-b]pyridin-3-yl}pyrazin-2-yl)-N-(2,2,2-trifluoroethyl)azetidine-2-carboxamide

To a vial were added the product of Step 4 (15.1 mg, 0.036 mmol), 5-chloro-1-[(4-methylphenyl)sulfonyl]-3-(4,4,5,5-tetramethyl-1,3,2-dioxaborolan-2-yl)-1H-pyrrolo[2,3-b]-pyridine (30.6 mg, 0.071 mmol), PdCl₂(dppf) (8 mg, 9.80 μmol), 2M Na₂CO₃ (0.06 ml, 0.120 mmol) and 1,4-dioxane (1 ml). The vial was vacuum/N2 purged 3 times. The mixture was heated at 80° C. for overnight, diluted with EtOAc and washed with water, brine, dried and concentrated to afford residue, which was purified on silica gel (0%-50% EtOAc, 20 column) to afford the title compound. MS APCI: [M+H]⁺ m/z 695.2.

### Step 6. (2S,3S)-1-[6-(5-chloro-1H-pyrrolo[2,3-b]pyridin-3-yl)pyrazin-2-yl]-3-hydroxy-N-2,2,2-trifluoroethyl)azetidine-2-carboxamide

To a vial were added the product of Step 5 (24 mg, 0.035 mmol), K₂CO₃ (21 mg, 0.152 mmol) and MeOH (1 ml). The mixture was heated at 50° C. for 111, evaporated and purified on silica gel (DCM/MeOH=9/1). Further purification on RP HPLC afforded the title compound. MS APCI: [M+H]⁺ m/z 427.1. ¹H NMR (500 MHz, CD₃OD) δ 8.74 (d, 1H), 8.38 (s, 1H), 8.24 (d, 2H), 8.05 (s, 1H), 5.30-5.26 (m, 1H), 4.71 (d, 1H), 4.61 (dd, 1H), 4.48 (d, 1H), 4.28 (d, 1H), 4.03-3.87 (m, 1H), 3.78 (dd, 1H), rhJAK3 IC50: +++

### Example 4

### (2R)-1-[6-(5-Chloro-1H-pyrrolo[2,3-b]pyridin-3-yl)pyrazin-2-yl]-4-methylsulfonyl-N-(2,2,2-trifluoroethyl)piperazine-2-carboxamide

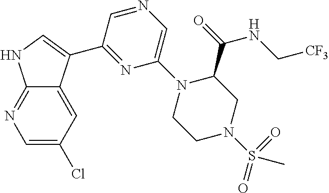 [SMILES: CS(=O)(=O)N1CCN(c2cncc(-c3c[nH]c4ncc(Cl)cc34)n2)[C@@H](C(=O)NCC(F)(F)F)C1]

### Step 1. 1-benzyl 4-tert-butyl (2R)-2-[(2,2,2-trifluoroethyl)carbamoyl]piperazine-1,4-dicarboxylate

A solution of (R)—N4-BOC-N-1-CBZ-piperazine carboxylic acid (4.0 g, 11 mmol), 2,2,2-trifluoroethylamine hydrochloride (1.78 g, 13 mmol), EDC (2.74 g, 14 mmol), HOBt (1.68 g, 11 mmol) and Hunig's base (5.75 mL, 33 mmol) in DCM (100 mL) was stirred at ambient temperature for 18 h. The reaction mixture was washed with aqueous 5% sodium bicarbonate (100 mL) and brine. The organic layer was dried over sodium sulfate, filter and concentrated in vacuo. The residue was purified by silica gel chromatography, eluting with a gradient of 5-70% ethyl acetate in hexane, to afford the title compound as white foam. MS APCI: [M+Na]⁺ m/z 468.0.

### Step 2. tert-butyl (3R)-3-[(2,2,2-trifluoroethyl)carbamoyl]piperazine-1-carboxylate

A flask containing the solution of the product of Step 1 (2.88 g, 6.47 mmol) in methanol (60 mL) was evacuated with vacuum and purged with nitrogen. 10% Pd/C (0.9 g) was added to the solution and the resulting mixture was purged with nitrogen again. The flask was then evacuated with vacuum and filled with hydrogen gas via a balloon and the mixture was stirred for 1 h. The hydrogen atmosphere was exchanged for a nitrogen atmosphere, and the mixture was filtered through a celite cake, which was then washed with methanol (with 1% conc. NH4OH). The filtrate was concentrated in vacuo to yield the title compound which was used directly in the subsequent step without purification.

### Step 3. tert-butyl (3R)-4-(6-chloropyrazin-2-yl)-3-[(2,2,2-trifluoroethyl)carbamoyl]piperazine-1-carboxylate

A solution of the product of Step 2 (0.5 g, 1.6 mmol), 2,6-dichloropyrazine (0.29 g, 1.9 mmol) and triethylamine (0.56 mL, 4.0 mmol) in DMF (8 mL) was microwaved at 170° C. for 1 h and then heated at 100° C. for 18 h in an oil bath. The reaction mixture was concentrated in vacuo and the residue was purified by silica gel chromatography, with a gradient of 10-70% ethyl acetate in hexane to afford the title compound. MS APCI: [M+H]⁺ m/z=424.1.

### Step 4. tert-butyl (3R)-4-(6-{5-chloro-1-[(4-methylphenyl)sulfonyl]-1H-pyrrolo[2,3-b]pyridin-3-yl}pyrazin-2-yl)-3-[(2,2,2-trifluoroethyl)carbamoyl]piperazine-1-carboxylate

A solution of the product of Step 3 (0.15 g, 0.35 mmol), 5-chloro-1-[(4-methyl-phenyl)sulfonyl]-3-(4,4,5,5-tetramethyl-1,3,2-dioxaborolan-2-yl)-1H-pyrrolo[2,3-b]pyridine (0.17 g, 0.39 mmol), PdCl₂(dppf) (0.05 g, 0.07 mmol) and Na₂CO₃ (0.53 mL, 2 M in water, 1.06 mmol) in DMF (4 mL) was microwaved at 120° C. for 20 min. The reaction mixture was concentrated in vacuo and the residue was partitioned between ethyl acetate and aqueous 5% sodium bicarbonate. The organic layer was then washed with 1/2 brine and brine before dried over sodium sulfate, filtered and concentrated in vacuo. The crude product was purified by silica gel chromatography, eluting with a gradient of 1-5% methanol in DCM to afford the title compound. MS APCI: [M+H]⁺ m/z=694.2.

### Step 5. (2R)-1-(6-{5-chloro-1 [(4-methylphenyl)sulfonyl]-1H-pyrrolo[2,3-b]pyridin-3-yl}-pyrazin-2-yl)-N-(2,2,2-trifluoroethyl)piperazine-2-carboxamide

Into a flask containing the product of Step 4 (100 mg, 0.14 mmol) was added a cooled (0° C.) solution of ethyl acetate saturated with HCl gas. The reaction mixture was stirred at 0° C. for 30 min and concentrated in vacuo to give the title compound as a hydrochloride salt. MS APCI: [M+H]⁺ m/z=594.1.

### Step 6. (2R)-1-(6-{5-chloro-1-[(4-methylphenyl)sulfonyl]-1H-pyrrolo[2,3-b]pyridin-3-yl}-pyrazin-2-yl)-4-methylsulfonyl)-N-(2,2,2-trifluoroethyl)piperazine-2-carboxamide

Into a solution of the product of Step 5 (30 mg, 0.05 mmol) in DCM (1 mL) at 0° C. were added methanesulfonyl chloride (11 μL, 0.14 mmol) and triethylamine (26 μL, 0.19 mmol). After 6 d at 0° C., the reaction mixture was concentrated and the residue was used directly in the subsequent step without purification. MS APCI: [M+H]⁺ m/z=672.1.

### Step 7. (2R)-1-[6-(5-chloro-1H-pyrrolo[2,3-b]pyridin-3-yl)pyrazin-2-yl]-4-methylsulfonyl)-N-(2,2,2-trifluoroethyl)piperazine-2-carboxamide

Into a stirred suspension of the product of Step 6 (32 mg, 0.05 mmol) in MeOH (1 mL) was added sodium hydroxide (0.5 mL, 1 N in water, 0.5 mmol). The suspension was heated at 50° C. for 2 h, which became a solution. The reaction solution was cooled to ambient temperature and TFA was added dropwise until the solution was neutralized. The resulting solution was directly purified by reverse phase chromatography to yield the title compound as a TFA salt. MS APCI: [M+H]⁺ m/z=518.1. ¹H NMR (600 MHz, CD₃OD) δ 8.53 (d, J=2.4, 1H), 8.38 (s, 1H), 8.23 (d, J=2.4, 1H), 8.18 (s, 1H), 8.08 (s, 1H), 5.37 (bs, 1H), 4.34 (d, J=12.6, 1H), 4.26 (d, J=13.2, 1H), 4.00-3.88 (m, 2H), 3.77 (d, J=11.4, 1H), 3.67 (m, 1H), 3.13 (dt, J=11.4, 3.6, 1 H), 2.88 (s, 3H). rhJAK3 IC₅₀: +++

The following examples were prepared in an analogous manner of that described in Example 4 using materials that are commercially available or known, or that can be prepared using procedures known in the art or by generally following procedures described herein for various intermediates.

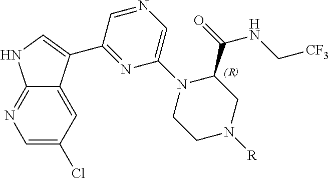

| | | | MS | rhJAK3 |
|---|---|---|---|---|
| Example | R | Chemical Name | [M + H]+ | IC₅₀ |
| 4-1 | H | (2R)-1-[6-(5-chloro-1H-pyrrolo[2,3-b]- pyridin-3-yl)pyrazin-2-yl]-N-(2,2,2- trifluoroethyl)piperazine-2-carboxamide | 439.1 | ++ |
| 4-2 | CH₃C(O)— | (2R)-4-acetyl-1-[6-(5-chloro-1H-pyrrolo- [2,3-b]pyridin-3-yl)pyrazin-2-yl]-N- (2,2,2-trifluoroethyl)piperazine-2- carboxamide | 481.1 | +++ |
| 4-3 | (CH₃)₃CC(O)— | (2R)-1-[6-(5-chloro-1H-pyrrolo[2,3-b]- pyridin-3-yl)pyrazin-2-yl]-4-(2,2- dimethylpropanoyl)-N-(2,2,2-trifluoro- ethyl)piperazine-2-carboxamide | 523.2 | ++ |

### Example 5

### 2-[3-(5-Chloro-1H-pyrrolo[2,3-b]pyridin-3-yl)-5H-pyrrolo[2,3-b]pyrazin-5-yl]-N-(2,2,2-trifluoroethyl)acetamide

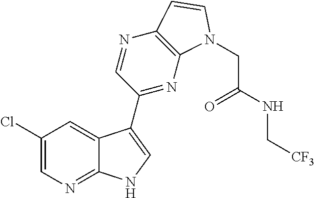 [SMILES: O=C(Cn1ccc2ncc(-c3c[nH]c4ncc(Cl)cc34)nc21)NCC(F)(F)F]

### Step 1. 2-(3-chloro-5H-pyrrolo[2,3-b]pyrazin-5-yl)-N-(2,2,2-trifluoroethyl)acetamide

To a solution of 3-chloro-5H-pyrrolo[2,3-b]pyrazine (50 mg, 0.326 mmol) in DMF (2 mL) at room temperature was slowly added sodium hydride (20.3 mg, 0.508 mmol). The reaction mixture was allowed to stir for 15 minutes. 2-Bromo-N-(2,2,2-trifluoroethyl)acetamide (88 mg, 0.40 mmol) was then added and the reaction mixture allowed to stir for 1 hour. Ethanol (0.1 mL) was added to the reaction mixture which was then loaded on to a silica cartridge and purified using medium pressure liquid chromatography (0-10%, methanol/dichloromethane) to yield the title compound. LC-MS (ES, m/z): 293.0 [M+H]⁺. ¹H-NMR (400 Hz, CDCl₃ ppm): 8.95 (1H, t, J=6.1 Hz), 8.47 (1H, s), 7.91 (1H, d, J=3.6 Hz), 6.72 (1H, d, J=3.6 Hz), 5.01 (2H, s), 3.93 (2H, m).

### Step 2. 2-(3-{5-chloro-1-[(4-methylphenyl)sulfonyl]-1H-pyrrolo[2,3-b]pyridin-3-yl}-5H-pyrrolo[2,3-b]pyrazin-5-yl)-N-(2,2,2-trifluoroethyl)acetamide

To a nitrogen gas purged mixture of 5-chloro-1-[(4-methylphenyl)sulfonyl]-3-(4,4,5,5-tetramethyl-1,3,2-dioxaborolan-2-yl)-1H-pyrrolo[2,3-b]pyridine (100 mg, 0.231 mmol), the product of Step 1 (80 mg, 0.273 mmol), and sodium bicarbonate (2M, 347 μl, 0.693 mmol) in DMF (2311 μl) was added PdCl₂-dppf (33.8 mg, 0.046 mmol). The reaction mixture was microwaved at 130° C. for 20 min., and ethyl acetate (3 mL) was added to the reaction mixture which was then loaded on to a silica cartridge and purified via medium pressure liquid chromatography (0-10%, dichloromethane/methanol) to yield the title compound. LC-MS (ES, m/z): 562.9 [M+H]⁺)

### Step 3. 2-[3-(5-chloro-1H-pyrrolo[2,3-b]pyridin-3-yl)-5H-pyrrolo[2,3-b]pyrazin-5-yl]-N-(2,2,2-trifluoroethyl)acetamide

The compound of Step 2 (28 mg, 0.050 mmol) was dissolved in a mixture of MeOH (400 μL) and THF (400 μL). Sodium hydroxide (1M, 0.6 mL, 0.600 mmol) was added to the mixture, which was allowed to stir for 3 hrs at room temperature then neutralized using hydrochloric acid (1M, 0.6 mL, 0.600 mmol), concentrated and purified directly by preparative HPLC reverse phase (C-18), eluting with acetonitrile/water+0.1% TFA (0-100%) to yield the title compound. LC-MS (ES, m/z): 409.1 [M+H]⁺. ¹H-NMR (400 Hz, CDCl₃, ppm): 12.3 (1H, s), 9.07 (1H, t, J=6.6 Hz), 9.01 (1H, s), 8.73 (1H, d, J=2.4 Hz), 8.49 (1H, d, J=2.8 Hz), 8.27 (1H, d, J=2.4 Hz), 7.75 (1H, d, J=3.6 Hz), 6.62 (1H, d, J=3.6 Hz), 5.10 (2H, s), 3.97 (2H, m). rhJAK3 IC₅₀: ++

### General Scheme for Example 6

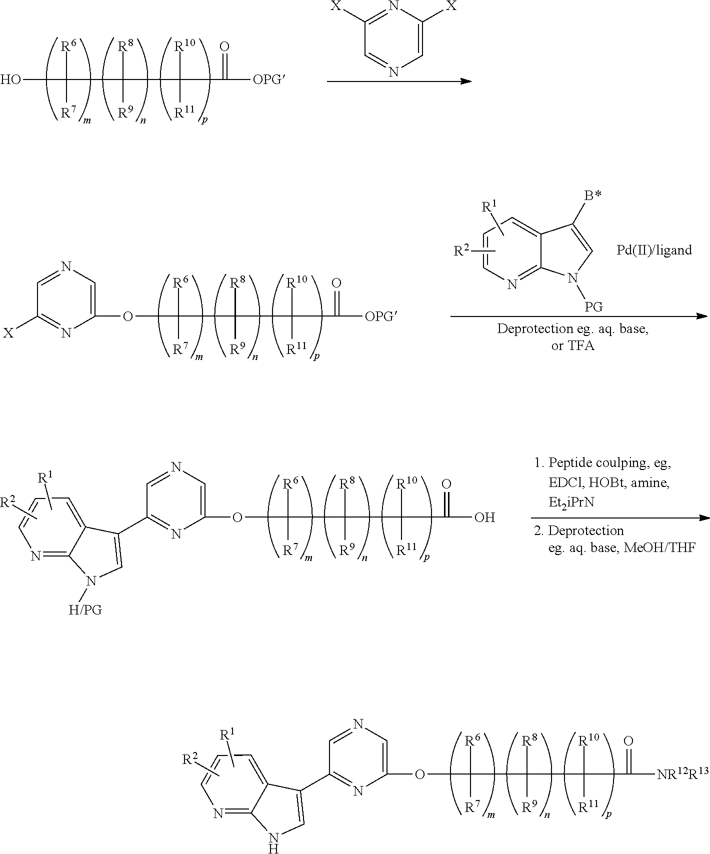

### Example 6

### 1-{[6-(5-Chloro-1H-pyrrolo[2,3-b]pyridin-3-yl)pyrazin-2-yl]oxy}-N-(2,2,2-trifluoroethyl)-cyclopropanecarboxamide

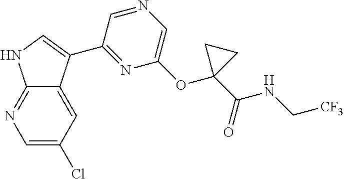 [SMILES: O=C(NCC(F)(F)F)C1(Oc2cncc(-c3c[nH]c4ncc(Cl)cc34)n2)CC1]

### Step 1. Meth 1-[6-chloropyrazin-2-yl)oxy]cyclopropanecarboxylate

Sodium hydride (0.258 g, 60% dispersion in mineral oil, 6.46 mmol) was added to a solution of methyl 1-hydroxycyclopropanecarboxylate (0.5 g, 4.31 mmol) in anhydrous DMF (14.4 mL) at 0° C. and the mixture was stirred for 30 min. 2,6-Dichloropyrazine (0.642 g, 4.31 mmol) was added and the resulting mixture was allowed to stir at 0° C. for additional 1 h before quenched by two drops of water. The reaction mixture was concentrated in vacuo and the residue was applied to silica gel chromatography, eluting with a gradient of ethyl acetate:hexane 5:95 to 50:50 to afford the title compound, which was contaminated with residual DMF. MS APCI: [M+H]⁺ m/z=229.0.

### Step 2. Methyl 1-[(6-{5-chloro-1-[(4-methylphenyl)sulfonyl]-1H-pyrrolo[2,3-b]pyridin-3-yl}-pyrazin-2-yl)oxy]cyclopropanecarboxylate

A solution of the product of Step 1 (0.21 g, 0.924 mmol), 5-chloro-1-[(4-methyl-phenyl)sulfonyl]-3-(4,4,5,5-tetramethyl-1,3,2-dioxaborolan-2-yl)-1H-pyrrolo[2,3-b]pyridine (0.4 g, 0.924 mmol), PdCl₂(dppf) (0.135 g, 0.185 mmol) and Na₂CO₃ (1.34 mL, 2 M in water, 2.77 mmol) in DMSO (9.2 mL) was microwaved at 125° C. for 20 min. The reaction mixture was concentrated in vacuo and the residue was partitioned between ethyl acetate and water. The organic layer was then washed with 1/2 brine and brine before dried over sodium sulfate, filtered and concentrated in vacuo. The crude product was purified by silica gel chromatography, eluting with a gradient of ethyl acetate:hexane 10:90 to 80:20 to afford the title compound. MS APCI: [M+H]⁺ m/z=499.1.

### Step 3. 1-{[6-(5-chloro-1H-pyrrolo[2,3-b]pyridin-3-yl)pyrazin-2-yl]oxy}cyclopropanecarboxylic acid

Into a stirred suspension of the product of Step 2 (0.2 g, 0.4 mmol) in methanol (4 mL) was added sodium hydroxide (2 mL, 1 N in water, 2 mmol). The suspension was heated at 50° C. for 5 h, which became a solution. The reaction solution was cooled to ambient temperature and methanol was evaporated. 1N aqueous solution of HCl was then added until pH reached 5. The precipitate was filtered and dried under vacuum to afford the title compound. MS APCI: [M+H]⁺ m/z=331.1.

### Step 4. 1-{[6-(5-chloro-1H-pyrrolo[2,3-b]pyridin-3-yl)pyrazin-2-yl]oxy}-N-(2,2,2-trifluoro-ethyl)cyclopropanecarboxamide

A solution of the product of Step 3 (20 mg, 0.06 mmol), 2,2,2-trifluoroethylamine (12 mg, 0.09 mmol), PyBop (63 mg, 0.12 mmol) and triethylamine (25 μL, 0.181 mmol) in DMF (0.6 mL) was stirred for 1 h. The reaction mixture was filtered and purified on reverse phase chromatography to afford the title compound as the trifluoroacetate salt. MS APCI: [M+H]⁺ m/z 412.1. ¹H NMR (600 MHz, CD₃OD) δ 8.77 (d, J=1.8, 1H), 8.70 (s, 1H), 8.25 (s, 1H), 8.21 (d, J=2.4, 1H), 8.01 (s, 1H), 3.85 (m, 2H), 1.70 (m, 2H), 1.32 (m, 2H). rhJAK3 IC₅₀: +++

The following examples were prepared in an analogous manner of that described in GENERAL SCHEME FOR EXAMPLE 6 using materials that are commercially available or known, or that can be prepared using procedures known in the art or by generally following procedures described herein for various intermediates.

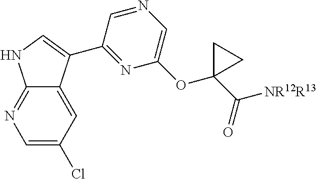

| | | | MS | rhJAK3 |
|---|---|---|---|---|
| Example | R¹²/R¹³ | Chemical Name | [M + H]+ | IC₅₀ |
| 6-1 | H/H | 1-{[6-(5-chloro-1H-pyrrolo[2,3-b]pyridin- 3-yl)pyrazin-2-yl]oxy}cyclopropane- carboxamide | 329.1 | +++ |
| 6-2 | CH₃/CH₃ | 1-{[6-(5-chloro-1H-pyrrolo[2,3-b]pyridin- 3-yl)pyrazin-2-yl]oxy}-N,N-dimethyl- cyclopropanecarboxamide | 357.1 | +++ |
| 6-3 | H/(CH₂)₂CF₃ | 1-{[6-(5-chloro-1H-pyrrolo[2,3-b]pyridin- 3-yl)pyrazin-2-yl]oxy}-N-(3,3,3-trifluoro- propyl)cyclopropanecarboxamide | 425.2 | + |

| | | | |
|---|---|---|---|
| 6-4 | 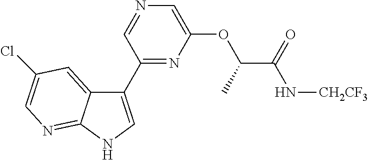 2(S) 2-{[6-(5-chloro-1H-pyrrolo[2,3-b]pyridin-3-yl)pyrazin- 2-yl]oxy}-N-(2,2,2-trifluoroethyl)propanamide | 400.1 | +++ |

### Example 7

### 6-(5-chloro-1H-pyrrolo[2,3-b]pyridin-3-yl)-N-(2,2,2-trifluoroethyl)pyrazine-2-carboxamide

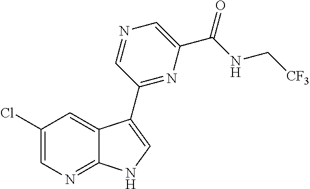 [SMILES: O=C(NCC(F)(F)F)c1cncc(-c2c[nH]c3ncc(Cl)cc23)n1]

### Step 1. Methyl 6-{5-chloro-1-[(4-methylphenyl)sulfonyl]-1H-pyrrolo[2,3-b]pyridin-3-yl}-pyrazine-2-carboxylate

A solution of methyl 6-chloropyrazine-2-carboxylate (0.1 g, 0.579 mmol), 5-chloro-1-[(4-methylphenyl)sulfonyl]-3-(4,4,5,5-tetramethyl-1,3,2-dioxaborolan-2-yl)-1H-pyrrolo[2,3-b]pyridine (0.276 g, 0.637 mmol), PdCl₂(dppf) (0.085 g, 0.116 mmol) and Na₂CO₃ (0.87 mL, 2 M in water, 1.74 mmol) in DMF (6 mL) was microwaved at 130° C. for 20 min. The reaction mixture was concentrated in vacuo and the residue was partitioned between ethyl acetate and water. The organic layer was then washed with 1/2 brine and brine before dried over sodium sulfate, filtered and concentrated in vacuo. The crude product was purified by silica gel chromatography, eluting with a gradient of ethyl acetate:hexane 10:90 to 80:20 to afford the title compound. MS APCI: [M+H]⁺ m/z=443.0.

### Step 2. 6-(5-chloro-1H-pyrrolo[2,3-b]pyridin-3-yl)pyrazine-2-carboxylic acid

Into a stirred suspension of the product of Step 1 (0.1 g, 0.226 mmol) in methanol (2 mL) was added aqueous solution of sodium hydroxide (2 mL, 1 N, 2.0 mmol). The suspension was heated at 50° C. for 2 h, which became a solution. The reaction solution was cooled to ambient temperature and methanol was evaporated. 1N aqueous solution of HCl was then added until pH reached 5. The precipitate was filtered and dried under vacuum to afford the title compound. MS APCI: [M+H]⁺ m/z=275.0.

### Step 3. 1-{[6-(5-chloro-1H-pyrrolo[2,3-b]pyridin-3-yl)-N-(2,2,2-trifluoroethyl)pyrazine-2-carboxamide

A solution of the product of Step 2 (55 mg, 0.20 mmol), 2,2,2-trifluoroethylamine (21 mg, 0.21 mmol), EDC (51 mg, 0.27 mmol), HOBt (32 mg, 0.21 mmol), triethylamine (87 μL, 0.62 mmol) in dichloromethane (2 mL) was stirred at ambient temperature for 18 h. The reaction mixture was directly loaded onto a silica gel column, and the product was eluted using a gradient of 1-10% methanol (containing 10% conc. NH₄OH) in DCM to provide the title compound. MS APCI: [M+H]⁺ m/z=356.0. 114 NMR (600 MHz, CD₃OD) δ 9.25 (s, 1H), 8.96 (s, 1H), 8.80 (d, J=1.8, 1H), 8.47 (s, 1H), 8.28 (d, J=2.4, 1H), 4.19 (q, J=9, 2H). rhJAK3 IC50: +

The following example were prepared in an analogous manner of that described in Example 7 using materials that are commercially available or known, or that can be prepared using procedures known in the art or by generally following procedures described herein for various intermediates.

| | | MS | rhJAK3 |
|---|---|---|---|
| Example | Structure/Chemical Name | [M + H]+ | IC₅₀ |
| 7-1 | 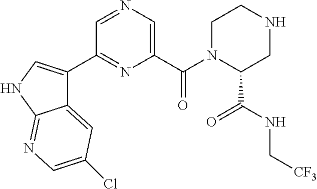 [SMILES: O=C(NCC(F)(F)F)[C@H]1CNCCN1C(=O)c1cncc(-c2c[nH]c3ncc(Cl)cc23)n1] (2R)-1-{[6-(5-chloro-1H-pyrrolo[2,3-b]pyridin-3-yl)- pyrazin-2-yl]carbonyl}-N-(2,2,2-trifluoroethyl)piperazine-2- carboxamide | 467.1 | + |

### Example 8

### 2-[6-(5-chloro-1H-pyrrolo[2,3-b]pyridin-3-yl)pyrazin-2-yl]-N-(2,2,2-trifluoroethyl)acetamide

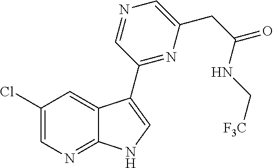 [SMILES: O=C(Cc1cncc(-c2c[nH]c3ncc(Cl)cc23)n1)NCC(F)(F)F]

### Step 1. diethyl (6-chloropyrazin-2-yl)propanedioate

To a suspension of sodium hydride (60 wt % in mineral oil) (1.074 g, 26.8 mmol) in THF (8 mL) at 0° C. was added diethyl malonate (4.10 ml, 26.8 mmol) in THF (8 mL). The solution was allowed to stir for 10 min before the addition of 2,6-dichloropyrazine (2 g, 13.42 mmol) in THF (8 mL). The mixture was then heated to reflux for 4 hours. The reaction mixture was diluted with ethyl acetate and washed with brine. The organic layer was back extracted once and the combined organic layers were dried over sodium sulfate, filtered and concentrated. The residue was purified by flash column chromatography on silica gel (EtOAc/hexanes gradient, 2-20%) to give diethyl (6-chloropyrazin-2-yl)propanedioate.

### Step 2. diethyl (6-{5-chloro-1-[(4-methylphenyl)sulfonyl]-1H-pyrrolo[2,3-b]pyridin-3-yl}-pyrazin-2-yl)propanedioate

To a solution of the product of Step 1 (595 mg, 1.527 mmol), potassium [5-chloro-1-(phenylsulfonyl)-1H-pyrrolo[2,3-b]pyridin-3-yl](trifluoro)borate (700 mg, 1.696 mmol), Ruphos (119 mg, 0.254 mmol) and palladium(II) chloride (15.04 mg, 0.085 mmol) in n-propanol (17 mL) was added TEA (0.709 mL, 5.09 mmol) The mixture was heated to 80° C. for 18 hrs. The reaction was then cooled, filtered and concentrated. The residue was purified by flash column chromatography on silica gel (EtOAc/hexanes gradient, 5-70%) to give desired product.

### Step 3. [6-(5-chloro-1H-pyrrolo[2,3-b]pyridin-3-yl)pyrazin-2-yl]acetic acid

The product of Step 2 (706 mg, 1300 mmol) and potassium carbonate (719 mg, 5.20 mmol) were suspended in a mixture of EtOH (11 mL) and water (5.5 mL). The reaction was heated to 80° C. and allowed to stir. After stirring for 5 hours after reaction allowed to cool, and concentrated to remove ethanol. The resulting solution was acidified with aqueous citric acid, during which solids precipitate out. The precipitate was collected through vacuum filtration and dried overnight on the high vacuum to give the desired product.

### Step 4. 2-[6-(5-chloro-1H-pyrrolo[2,3-b]pyridin-3-yl)pyrazin-2-yl]-N-(2,2,2-trifluoroethyl)-acetamide

The product of Step 3 (24.8 mg, 0.086 mmol), 2,2,2-trifluoroethylamine hydrochloride (12.81 mg, 0.094 mmol) and HOBT (19.73 mg, 0.129 mmol) were dissolved in DMF (1 mL) and TEA (0.048 mL, 0.344 mmol). EDC (19.76 mg, 0.103 mmol) was then added and the reaction mixture was heated to 80° C. for 3 h. The mixture was cooled, diluted with ethyl acetate, washed with aqueous sodium hydrogen carbonate and brine. Aqueous layers were back extracted once with ethyl acetate, the combined organic layers were dried with sodium sulfate, filtered and the solvent was evaporated under reduced pressure. The residue was purified by preparative HPLC Reverse phase (C-18), eluting with acetonitrile/water+0.025% TFA (eluting with 10-100 MeCN). The desired fractions were combined in a separatory funnel, diluted with a 3:1 CHCl₃:IPA solution and washed with a saturated solution of sodium bicarbonate. The organic layer was dried with sodium sulfate, filtered and the solvent was evaporated under reduced pressure to give product as a free base. ¹H NMR (600 MHz, DMSO) δ 12.46 (s, 1H), 9.05 (s, 1H), 8.94 (t, 1H), 8.73 (d, 1H), 8.53 (s, 1H), 8.31 (s, 1H), 8.28 (s, 1H), 4.04-3.89 (m, 2H), 3.83 (s, 2H); APCI: [M+H]⁺ m/z 370.1. rhJAK3 IC₅₀: ++

The following examples were prepared in an analogous manner of that described in Example 8 using materials that are commercially available or known, or that can be prepared using procedures known in the art or by generally following procedures described herein for various intermediates.

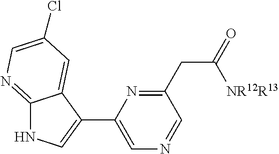

| | R¹²/R¹³ or | | MS | rhJAK3 |
|---|---|---|---|---|
| Example | NR¹²R¹³ | Chemical Name | [M + H]+ | IC₅₀ |
| 8-1 | 4-morpholinyl | 5-chloro-3-[6-(2-morpholin-4-yl-2-oxoethyl) pyrazin-2-yl]-1H-pyrrolo-[2,3-b]pyridine | 358.1 | + |
| 8-2 | H/CH₂CH₂CN | 2-[6-(5-chloro-1H-pyrrolo[2,3-b]-pyridin- 3-yl)pyrazin-2-yl]-N-(2-cyanoethyl) acetamide | 341.1 | + |
| 8-3 | H/CH₂CH₂CF₃ | 2-[6-(5-chloro-1H-pyrrolo[2,3-b]-pyridin- 3-yl)pyrazin-2-yl]-N-(3,3,3-trifluoropropyl) acetamide | 384.1 | ++ |
| 8-4 | H/CH₂CN | 2-[6-(5-chloro-1H-pyrrolo[2,3-b]-pyridin-3- yl)pyrazin-2-yl]-N-(cyanomethyl)acetamide | 327.0 | ++ |

### Example 9

### (2E)-3-[6-(5-chloro-1H-pyrrolo[2,3-b]pyridin-3-yl)pyrazin-2-yl]-2-methyl-N-(2,2,2-trifluoro-ethyl)prop-2-enamide

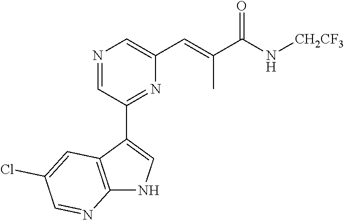

The title compound was prepared in an analogous manner of that described in Scheme 5 using Intermediate 2. MS [M+H]⁺: 396.1. rhJAK3 IC₅₀: ++

### Example 10

### 4-[6-(5-Chloro-1H-pyrrolo[2,3-b]pyridin-3-yl)pyrazin-2-yl]-N-(2,2,2-trifluoroethyl)benzamide

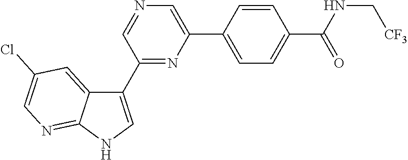 [SMILES: O=C(NCC(F)(F)F)c1ccc(-c2cncc(-c3c[nH]c4ncc(Cl)cc34)n2)cc1]

The title compound was prepared in an analogous manner of that described in Scheme 3 using Intermediate 3. Mass [M+H]+: 432.0. rhJAK3 IC₅₀: +

The following examples were prepared in an analogous manner of that described in Scheme 3 using materials that are commercially available or known, or that can be prepared using procedures known in the art or by generally following procedures described herein for various intermediates.

| | | MS | rhJAK3 |
|---|---|---|---|
| Example | Structure/Chemical Name | [M + H]+ | IC₅₀ |
| 10-1 | 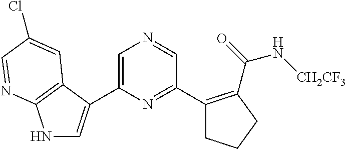 2-[6-(5-chloro-1H-pyrrolo[2,3-b]pyridin-3-yl)pyrazin-2-yl]- N-(2,2,2-trifluoroethyl)cyclopent-1-ene-1-carboxamide | 422.1 | +++ |
| 10-2 | 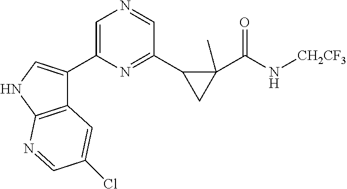 2-[6-(5-chloro-1H-pyrrolo[2,3-b]pyridin-3-yl)pyrazin-2-yl]- 1-methyl-N-(2,2,2-trifluoroethyl)cyclopropanecarboxamide | 410.1 | +++ |

### Example 11

### 3-[6-(5-chloro-1H-pyrrolo[2,3-b]pyridin-3-yl)pyrazin-2-yl]-2-methyl-N-(2,2,2-trifluoroethyl)-propanamide

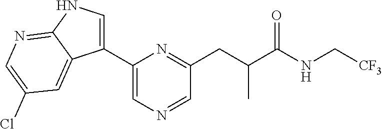 [SMILES: CC(Cc1cncc(-c2c[nH]c3ncc(Cl)cc23)n1)C(=O)NCC(F)(F)F]

The title compound was prepared in an analogous manner of that described in Scheme 6 using Intermediate 4. [M+H]+: 398.1. rhJAK3 IC₅₀: ++

### Example 12

### 3-[6-(5-chloro-1H-pyrrolo[2,3-b]pyridin-3-yl)pyrazin-2-yl]-N-(2,2,2-trifluoroethyl)propanamide

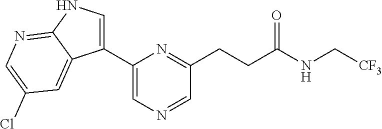 [SMILES: O=C(CCc1cncc(-c2c[nH]c3ncc(Cl)cc23)n1)NCC(F)(F)F]

The title compound was prepared in an analogous manner of that described in Scheme 6 and for Intermediate 4. [M+H]+: 384.0. rhJAK3 IC₅₀: ++

### Example 13

N²-[6-(5-cyclopropyl-1H-pyrrolo[2,3-b]pyridin-3-yl)pyrazin-2-yl]-N-(2,2,2-trifluoroethyl)-D-alaninamide

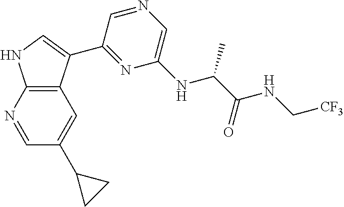 [SMILES: C[C@@H](Nc1cncc(-c2c[nH]c3ncc(C4CC4)cc23)n1)C(=O)NCC(F)(F)F]

### Step 1. N²-(6-{5-cyclopropyl-1-[(4-methylphenyl)sulfonyl]-1H-pyrrolo[2,3-b]pyridin-3-yl)pyrazin-2-yl]-N-(2,2,2-trifluoroethyl)-D-alaninamide

A solution of N²-(6-{5-chloro-1-[(4-methylphenyl)sulfonyl]-1H-pyrrolo[2,3-b]pyridin-3-yl}pyrazin-2-yl)-N-(2,2,2-trifluoroethyl)-D-alaninamide (Example 1, Step 4, 60 mg, 0.11 mmole), cyclopropylboronic acid pinacol ester (37 mg, 0.22 mmol), x-phos (21 mg, 0.04 mmole), palladium(II) acetate (5 mg, 0.02 mmol), potassium phosphate tribasic (0.85 mL, 0.17 mmol) in 2-methyl THF (1 mL) in a microwave vial was degassed using vacuum and filled with nitrogen. The solution was microwaved at 1200 for 20 min. The resulting mixture was filtered and the filtrate was concentrated. The crude product was used directly without purification. MS APCI: [M+H]⁺ m/z=559.2.

### Step 2. N²-[6-(5-cyclopropyl-1H-pyrrolo[2,3-b]pyridin-3-yl)pyrazin-2-yl]-N-(2,2,2-trifluoroethyl)-D-alaninamide.TFA

The tosyl group of the product of Step 1 was removed via the previously described method in Example 1, Step 5 to afford the title compound. MS APCI: [M+H]⁺ m/z=405.2. ¹H NMR (600 MHz, CD₃OD) δ 8.78 (s, 1H), 8.29 (s, 1H), 8.21 (s, 1H), 8.11 (s, 1H), 7.84 (s, 1H), 4.51 (q, J=9 Hz, 1H), 3.855 (m, 214), 2.27 (m, 1H), 1.58 (d, J=9 Hz, 1H), 1.13 (d, J=9.6 Hz, 2H), 0.90 (m, 2H). rhJAK3 IC₅₀: ++++

### Example 14

N²-{6-[5-(1-methyl-1H-pyrazol-4-yl)-1H-pyrrolo[2,3-b]pyridin-3-yl]pyrazin-2-yl}-N-(2,2,2-trifluoroethyl)-D-alaninamide

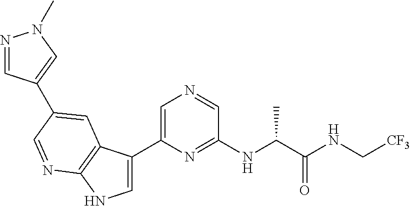 [SMILES: C[C@@H](Nc1cncc(-c2c[nH]c3ncc(-c4cnn(C)c4)cc23)n1)C(=O)NCC(F)(F)F]

N²-(6-{5-chloro-1-[(4-methylphenyl)sulfonyl]-1H-pyrrolo[2,3-b]pyridin-3-yl}-pyrazin-2-yl)-N-(2,2,2-trifluoroethyl)-D-alaninamide (51 mg, 0.092 mmol), 1-methyl-4-(4,4,5,5-tetramethyl-1,3,2-dioxaborolan-2-yl)-1H-pyrazole (42 mg, 0.202 mmol), X-Phos (17.2 mg, 0.036 mmol), palladium(II) acetate (5.5 mg, 0.024 mmol), 2-MeTHF (1.2 ml) then potassium phosphate tribasic (0.6 ml, 0.120 mmol) were added to a vial. The mixture was vacuumed then N₂ purged 3 times and heated at 50° C. for overnight, filtered and concentrated to afford residue, which was used in next step directly to remove the tosyl group. MS [M+H]+: 445.1. rhJAK3 IC50: ++++

### Example 15

N₂-[6-(5-methyl-1H-pyrrolo[2,3-b]pyridin-3-yl)pyrazin-2-yl]-N-(2,2,2-trifluoroethyl)-D-alaninamide

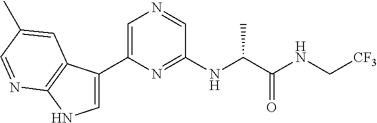 [SMILES: Cc1cnc2[nH]cc(-c3cncc(N[C@H](C)C(=O)NCC(F)(F)F)n3)c2c1]

A Biotage microwave vial was charged with N²-(6-{5-chloro-1-[(4-methyl-phenyl)sulfonyl]-1H-pyrrolo[2,3-h]pyridin-3-yl}pyrazin-2-yl)-N-(2,2,2-trifluoroethyl)-D-alaninamide (60 mg, 0.109 mmol), potassium trifluoro(methyl)borate(1-) (16 mg, 0.131 mmol), RuPhos (24.6 mg, 0.053 mmol), Pd(OAc)₂ (6.4 mg, 0.029 mmol) and K₂CO₃ (47.4 mg, 0.343 mmol). The test tube was sealed with a cap, evacuated and purged with N₂ three times. To the vial were added toluene (1 ml) and water (0.100 ml) and the vial was heated under microwave at 150° C. for 30 min. The mixture was filtered and concentrated to afford residue, which was used in next step directly to remove the tosyl group. MS [M+H]+: 3792. rhJAK3 IC50: +++

The following examples were prepared in an analogous manner of that described in Scheme 7 using materials that are commercially available or known, or that can be prepared using procedures known in the art or by generally following procedures described herein for various intermediates.

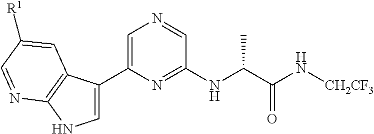

| | | | MS | JAK3 |
|---|---|---|---|---|
| Example | R¹ | Chemical Name | [M + H]+ | IC50 |
| 15-1 | 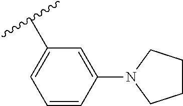 [SMILES: *c1cccc(N2CCCC2)c1] | N²-{6-[5-(3-pyrrolidin-1-ylphenyl)-1H- pyrrolo[2,3-b]pyridin-3-yl]pyrazin-2- yl}-N-(2,2,2-trifluoroethyl)-D- alaninamide | 510.3 | ++++ |
| 15-2 | 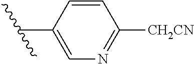 | N²-(6-{5-[6-(cyanomethyl)pyridin-3- yl]-1H-pyrrolo[2,3-b]pyridin-3-yl}- pyrazin-2-yl)-N-(2,2,2-trifluoroethyl)-D- alaninamide | 481.2 | ++++ |
| 15-3 | 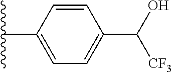 [SMILES: *c1ccc(C(O)C(F)(F)F)cc1] | N-(2,2,2-trifluoroethyl)-N²-(6-{5-[4- (2,2,2-trifluoro-1-hydroxyethyl)phenyl]- 1H-pyrrolo[2,3-b]pyridin-3-yl}pyrazin- 2-yl)-D-alaninamide | 539.2 | ++++ |
| 15-4 | 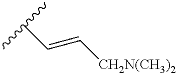 | N²-(6-{5-[(1E)-3-(dimethylamino)prop- 1-en-1-yl]-1H-pyrrolo[2,3-b]pyridin-3- yl}pyrazin-2-yl)-N-(2,2,2-trifluoro- ethyl)-D-alaninamide | 448.3 | +++ |
| 15-5 | 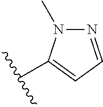 [SMILES: *c1ccnn1C] | N²-{6-[5-(1-methyl-1H-pyrazol-5-yl)- 1H-pyrrolo[2,3-b]pyridin-3-yl]pyrazin- 2-yl}-N-(2,2,2-trifluoroethyl)-D- alaninamide | 445.2 | +++ |
| 15-6 | 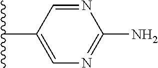 [SMILES: *c1cnc(N)nc1] | N²-{6-[5-(2-aminopyrimidin-5-yl)-1H- pyrrolo[2,3-b]pyridin-3-yl]pyrazin-2- yl}-N-(2,2,2-trifluoroethyl)-D- alaninamide | 458.2 | ++++ |
| 15-7 | 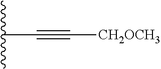 | N²-{6-[5-(3-methoxyprop-1-yn-1-yl)- 1H-pyrrolo[2,3-b]pyridin-3-yl]pyrazin- 2-yl}-N-(2,2,2-trifluoroethyl)-D- alaninamide | 433.2 | ++++ |
| 15-8 | 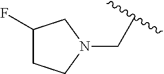 [SMILES: *CN1CCC(F)C1] | N²-(6-{5-[(3-fluoropyrrolidin-1-yl)- methyl]-1H-pyrrolo[2,3-b]pyridin-3-yl}- pyrazin-2-yl)-N-(2,2,2-trifluoroethyl)-D- alaninamide | 466.2 | ++ |
| 15-9 | 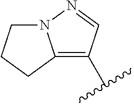 [SMILES: *c1cnn2c1CCC2] | N²-{6-[5-(5,6-dihydro-4H-pyrrolo[1,2- b]pyrazol-3-yl)-1H-pyrrolo[2,3-b]- pyridin-3-yl]pyrazin-2-yl}-N-(2,2,2- trifluoroethyl)-D-alaninamide | 471.1 | ++++ |
| 15-10 | 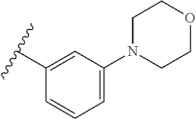 [SMILES: *c1cccc(N2CCOCC2)c1] | N²-{6-[5-(3-morpholin-4-ylphenyl)-1H- pyrrolo[2,3-b]pyridin-3-yl]pyrazin-2- yl}-N-(2,2,2-trifluoroethyl)-D- alaninamide | 526.2 | ++++ |
| 15-11 | 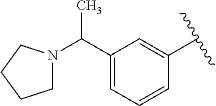 [SMILES: *c1cccc(C(C)N2CCCC2)c1] | N²-(6-{5-[3-(1-pyrrolidin-1-ylethyl)- phenyl]-1H-pyrrolo[2,3-b]pyridin-3- yl}pyrazin-2-yl)-N-(2,2,2- trifluoroethyl)-D-alaninamide | 538.2 | ++++ |
| 15-12 | 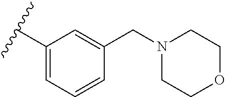 [SMILES: *c1cccc(CN2CCOCC2)c1] | N²-(6-{5-[3-(morpholin-4-yl- methyl)phenyl]-1H-pyrrolo[2,3-b]- pyridin-3-yl}pyrazin-2-yl)-N-(2,2,2- trifluoroethyl)-D-alaninamide | 540.1 | ++++ |
| 15-13 | 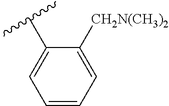 | N²-[6-(5-{2-[(dimethylamino)methyl]- phenyl}-1H-pyrrolo(2,3-b]pyridin-3- yl)pyrazin-2-yl]-N-(2,2,2-trifluoroethyl)- D-alaninamide | 498.2 | +++ |
| 15-14 | 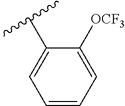 [SMILES: *c1ccccc1OC(F)(F)F] | N-(2,2,2-trifluoroethyl)-N²-(6-{5-[2- (trifluoromethoxy)phenyl]-1H- pyrrolo[2,3-b]pyridin-3-yl}pyrazin-2- yl)-D-alaninamide | 525.1 | ++ |
| 15-15 | 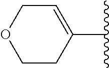 [SMILES: *C1=CCOCC1] | N²-{6-[5-(3,6-dihydro-2H-pyran-4-yl)- 1H-pyrrolo[2,3-b]pyridin-3-yl]pyrazin- 2-yl}-N-(2,2,2-trifluoroethyl)-D- alaninamide | 447.2 | ++++ |
| 15-16 | 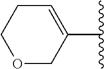 [SMILES: *C1=CCCOC1] | N²-{6-[5-(5,6-dihydro-2H-pyran-3-yl)- 1H-pyrrolo[2,3-b]pyridin-3-yl]pyrazin- 2-yl}-N-(2,2,2-trifluoroethyl)-D- alaninamide | 447.2 | ++++ |
| 15-17 | 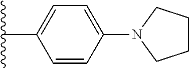 [SMILES: *c1ccc(N2CCCC2)cc1] | N²-{6-[5-(4-pyrrolidin-1-ylphenyl)-1H- pyrrolo[2,3-b]pyridin-3-yl]pyrazin-2- yl}-N-(2,2,2-trifluoroethyl)-D- alaninamide | 510.2 | ++++ |
| 15-18 | 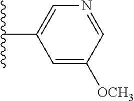 [SMILES: *c1cncc(OC)c1] | N²-{6-[5-(5-methoxypyridin-3-yl)-1H- pyrrolo[2,3-b]pyridin-3-yl]pyrazin-2- yl}-N-(2,2,2-trifluoroethyl)-D- alaninamide | 472.1 | ++++ |
| 15-19 | 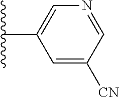 [SMILES: *c1cncc(C#N)c1] | N²-{6-[5-(5-cyanopyridin-3-yl)-1H- pyrrolo[2,3-b]pyridin-3-yl]pyrazin-2- yl}-N-(2,2,2-trifluoroethyl)-D- alaninamide | 467.1 | ++++ |
| 15-20 | 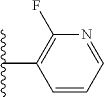 [SMILES: *c1cccnc1F] | N²-{6-[5-(2-fluoropyridin-3-yl)-1H- pyrrolo[2,3-b]pyridin-3-yl)pyrazin-2- yl}-N-(2,2,2-trifluoroethyl)-D- alaninamide | 460.1 | ++++ |

| | | | |
|---|---|---|---|
| 15-21 | 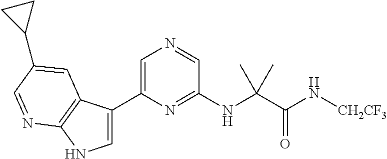 N2-[6-(5-cyclopropyl-1H-pyrrolo[2,3-b]pyridin-3-yl)pyrazin- 2-yl]-2-methyl-N-(2,2,2-trifluoroethyl)alaninamide | 419.2 | ++++ |

### Example 16

### 3-[6-({(2R)-3-methyl-1-oxo-1-[(2,2,2-trifluoroethyl)amino]butan-2-yl}amino)pyrazin-2-yl]-1H-pyrrolo[2,3-b]pyridine-5-carboxylic acid

### 3-[6-({(2R)-3-methyl-1-oxo-1-[(2,2,2-trifluoroethyl)amino]butan-2-yl}amino)pyrazin-2-yl]-1H-pyrrolo[2,3-b]pyridine-5-carboxamide.TFA

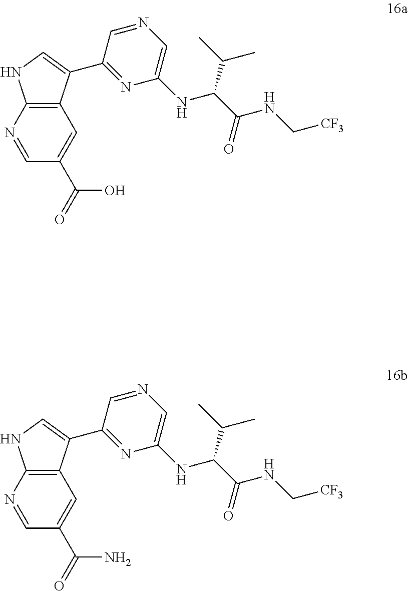 [SMILES: CC(C)[C@@H](Nc1cncc(-c2c[nH]c3ncc(C(=O)O)cc23)n1)C(=O)NCC(F)(F)F ; CC(C)[C@@H](Nc1cncc(-c2c[nH]c3ncc(C(N)=O)cc23)n1)C(=O)NCC(F)(F)F]

### Step 1. methyl 1H-pyrrolo[2,3-b]pyridine-5-carboxylate.HCl

A solution of 1H-pyrrolo[2,3-b]pyridine-5-carboxylic acid (0.5 g, 3.1 mmol) and HCl (10 mL, 4 M in water, 40 mmol) in methanol (25 mL) was heated to reflux for 16 h. The solution was concentrated and the residue was used for the next step without purification.

### Step 2. methyl 1-(phenylsulfonyl)-1H-pyrrolo[2,3-b]pyridine-5-carboxylate

Into a solution of the product of Step 1 (0.75 g, 3 mmol) in DMF (15 mL) at 0° C. was added sodium hydride (60%, 0.48 g, 12 mmol). After 5 min of stirring, benzenesulfonyl chloride (0.5 mL, 3.76 mmol) was added, and the resulting mixture was allowed to warm to ambient temperature and stirred for 16 h. The reaction was quenched by addition of water, and then concentrated in vacuo to dryness. The residue was applied to silica gel chromatography, eluting with a gradient of ethyl acetate:hexane 10:90 to 50:50 to yield the title compound (0.88 g, 92% yield). MS APCI: [M+H]⁺ m/z=331.0.

### Step 3. methyl 3-bromo-1-(phenylsulfonyl)-1H-pyrrolo[2,3-b]pyridine-5-carboxylate

Into a solution of the product of Step 2 (0.7 g, 2.2 mmol) in THF (15 mL) was added N-bromosuccinimide (0.47 g, 2.7 mmol). The resulting mixture was allowed to stir at ambient temperature for 16 h. The mixture was then concentrated in vacuo and the residue was purified by silica gel chromatography, eluting with a gradient of ethyl acetate:hexane 0:100 to 50:50 to afford the title compound (0.5 g, 57% yield). MS APCI: [M+H]⁺ m/z=408.9, 410.9.

### Step 4. methyl 1-(phenylsulfonyl)-3-(4,4,5,5-tetramethyl-1,3,2-dioxaborolan-2-yl)-1H-pyrrolo[2,3-b]pyridine-5-carboxylate

A microwave vial containing the product of Step 3 (0.5 g, 1.27 mmol), bis(pinacolato)diboron (0.51 g, 2.02 mmol), tricyclohexylphosphine (43 mg. 0.15 mmol), tris(dibenzylideneacetone)dipalladium(0) (58 mg, 0.06 mmol), potassium acetate (310 mg, 3.16 mmol) was degassed using vacuum and filled with nitrogen. Dioxane (15 mL) was added to the vial and the resulting mixture was again degassed with vacuum and filled with nitrogen. The mixture was then heated at 100° C. for 16 h, and diluted with ethyl acetate. The solution was filtered through celite and the filtrate was washed with aqueous ammonium chloride before being dried over sodium sulfate, filtered and concentrated in vacuo. The residue was used for the next step without purification.

### Step 5. potassium trifluoro[5-(methoxycarbonyl)-1-(phenylsulfonyl)-1H-pyrrolo[2,3-b]pyridine-3-yl]borate

Into a solution of the product of Step 4 (560 mg, 1.27 mmol) in acetone (15 mL) and water (6 mL) was added potassium hydrogen fluoride (593 mg, 7.6 mmol). The resulting mixture was stirred at ambient temperature for 5 h before concentrated in vacuo. The residue was diluted with acetone and concentrated again to dryness. The residue was then triturated with hot acetone (55° C.), and the remaining inorganics was filtered off. The filtrate was concentrated to be about 2 mL, and diethyl ether was added to the solution to promote precipitation. The resulting solution was cool to 0° C. for 30 min, and the precipitate was filtered and washed with diethyl ether to yield the desired product.

### Step 6: methyl 3-[6-({(2R)-3-methyl-1-oxo-1-[(2,2,2-trifluoroethyl)amino]butan-2-yl}amino)-pyrazin-2-yl]-1-(phenylsulfonyl)-1H-pyrrolo[2,3-b]pyridine-5-carboxylate

A solution of the product of Step 5 (90 mg, 0.21 mmol), N²-(6-chloropyrazin-2-yl)-N-(2,2,2-trifluoroethyl)-D-valinamide (66 mg, 0.21 mmol), palladium (II) chloride (5.7 mg, 0.03 mmol), Ru-Phos (45 mg, 0.10 mmol), and triethylamine (89 μL, 0.64 mmol) in n-propanol (2.1 mL) was degassed with vacuum and filled with nitrogen. The reaction mixture was heated at 100° C. for 16 h, and partitioned between ethyl acetate and aqueous 5% sodium bicarbonate. The organic layer was washed with brine, dried over sodium sulfate, filtered, and concentrated. The residue was subjected silica gel chromatography, eluting with a gradient of ethyl acetate:hexane 10:90 to 100:0 to afford the title compound. MS APCI: [M+H]⁺ m/z 591.1.

### Step 7: 3-[6-({(2R)-3-methyl-1-oxo-1-[(2,2,2-trifluoroethyl)amino]butan-2-yl}amino)pyrazin-2-yl]-1H-pyrrolo[2,3-b]pyridine-5-carboxylic acid

The solution of the product of Step 6 (46 mg, 0.08 mmol) and sodium hydroxide (1N, 0.39 mL, 0.39 mmol) in methanol (0.8 mL) was heated at 50° C. for 1 h. The solution was then acidified by addition of TFA and purified on reverse phase chromatography to afford the title compound. APCI: [M+H]⁺ m/z=436.1. rhJAK3 IC₅₀: +

### Step 8: 3-[6-({(2R)-3-methyl-1-oxo-1-[(2,2,2-trifluoroethyl)amino]butan-2-yl}amino)pyrazin-2-yl]-1H-pyrrolo[2,3-b]pyridine-5-carboxamide.TFA

A solution of the product of Step 7 (6.0 mg, 0.014 mmol) and 1,1′-carbonyldiimidazole (4.5 mg, 0.03 mmol) in DMF (0.5 mL) was stirred for 1 h at ambient temperature. Ammonium hydroxide (10 μL, 0.07 mmol) was added and the resulting mixture was allowed to stir for 16 h at ambient temperature. The mixture was then acidified by addition of TFA and purified on reverse phase chromatography to afford the title compound. MS APCI: [M+H]⁺ m/z=436.1. ¹H NMR (600 MHz, CD₃OD) δ 9.31 (d, J=2.4 Hz, 1H), 8.86 (d, J=1.8 Hz, 1H), 8.59 (t, J=6.6 Hz, 1H), 8.30 (s, 1H), 8.24 (s, 1H), 7.86 (s, 1H), 4.36 (d, J=6 Hz, 1H), 3.80 (m, 2H), 2.24 (m, 1H), 1.14 (dd, J=13.8 Hz, J=7.2 Hz, 6H). rhJAK3 IC₅₀: ++++

The following examples were prepared in an analogous manner of that described in Example 16 using materials that are commercially available or known, or that can be prepared using procedures known in the art or by generally following procedures described herein for various intermediates.

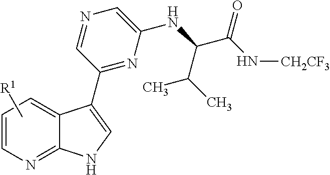

| | | | MS | JAK3 |
|---|---|---|---|---|
| Example | R¹ | Chemical Name | [M + H]+ | IC50 |
| 16-1 | 5-F | N²-[6-(5-fluoro-1H-pyrrolo[2,3-b]pyridin- 3-yl)pyrazin-2-yl]-N-(2,2,2-trifluoroethyl)- D-valinamide | 411.1 | +++ |
| 16-2 | 5-CN | N²-[6-(5-cyano-1H-pyrrolo[2,3-b]pyridin- 3-yl)pyrazin-2-yl]-N-(2,2,2-trifluoroethyl)- D-valinamide | 418.1 | ++++ |
| 16-3 | 5-CO₂CH₃ | Methyl 3-[6-({(2R)-3-methyl-1-oxo-1- [(2,2,2-trifluoroethyl)amino]butan-2-yl}- amino)pyrazin-2-yl]-1H-pyrrolo[2,3-b]- pyridine-5-carboxylate | 451.1 | +++ |

| | | | |
|---|---|---|---|
| 16-5 | 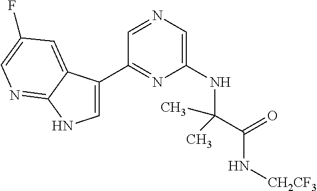 N²-[6-(5-fluoro-1H-pyrrolo[2,3-b]pyridin-3-yl)pyrazin-2-yl]-2- methyl-N-(2,2,2-trifluoroethyl)alaninamide | 427.1 | ++++ |

What is claimed is:

1. A compound of formula I: 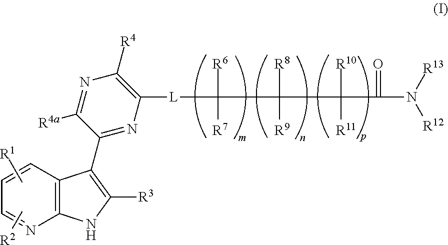
and pharmaceutically acceptable salts thereof; wherein
L is a bond, phenylene, O or NR⁵;
m is 0 or 1; n is 0, 1 or 2; p is 0 or 1; with the proviso that when L is O or NR⁵ m+n+p is 1 to 4;
R¹ and R² are each independently selected from the group consisting of: (1) H, (2) hydroxy, (3) cyano, (4) halogen, (5) C₁₋₆alkyl, C₂₋₁₀alkenyl, C₂₋₁₀alkynyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano, NR^{b}R^{c}, NHC(O)C₁₋₆alkyl and S(O)₂C₁₋₆alkyl, (6) CO₂R^{a}, (7) nitro, (8) NR^{b}R^{c}, (9) CONR^{b}R^{c}, (10) NR^{b}(CONR^{b}R^{c}), (11) S(O)₂NR^{b}R^{c} and (12) a ring containing moiety which is: aryl, aryloxy, arylC₁₋₆alkyl, arylcarbonyl, cycloalkyl, a heterocycle, a heteroaryl, any of which ring is optionally substituted with 1 to 5 groups independently selected from R^{z};
R³ is H or C₁₋₃alkyl;
R⁴ and R^{4a} are each independently selected from the group consisting of (1) hydrogen, (2) hydroxy, (3) cyano, (4) halogen, (5) C₁₋₆alkyl, C₂₋₁₀alkenyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano and NR^{b}R^{c}, (6) CO₂R^{a}, (7) nitro, (8) NR^{b}R^{c}, and (9) CONR^{b}R^{c}; or
when L is NR⁵, R⁴ and R⁵ together complete a pyrrole ring;
R⁵-R¹³ are each independently selected from the group consisting of (1) H, (2) C₁₋₆alkyl optionally substituted with 1 to 5 groups independently selected from halogen, CN, OR^{a}, CO₂R^{a}, CONR^{b}R^{c}, NR^{b}R^{c}, C₃₋₆cycloalkyl, optionally substituted aryl and optionally substituted heteroaryl wherein the optional substituents for aryl and heteroaryl are 1 to 3 groups independently selected from OR^{a}, NR^{b}R^{c}, halogen, C₁₋₆alkyl and C₁₋₆haloalkyl, (3) C₃₋₆cycloalkyl and (4) 2-azepinone; or
R⁶ and R¹⁰ together complete a cycloalkyl, cycloalkenyl or heterocyclyl, each of which is optionally substituted with 1 to 5 groups independently selected from R^{y}; or
R⁸ and R⁹ together complete a cycloalkyl optionally substituted with 1 to 5 groups independently selected from R^{y}; or
when L is NR⁵, R⁵ and R¹⁰ together complete a heterocycle optionally substituted with 1 to 5 groups independently selected from R^{y}; or
when n is 2, two R⁸ groups on adjacent carbon atoms together form a bond; or two R⁸ groups and two R⁹ groups on adjacent carbon atoms together complete a cycloalkenyl, aryl or heteroaryl each or which is optionally substituted with 1 to 4 groups independently selected from hydroxy, cyano, halogen, NR^{b}R^{c}, C₁₋₆alkyl, C₁₋₆haloalkyl, C₁₋₆hydroxyalkyl, C₂₋₄₀alkenyl, C₁₋₆alkylcarbonyl, C₁₋₆alkoxy, C₁₋₆haloalkoxy and C₁₋₆alkoxycarbonyl; or
R¹², R¹³ and the nitrogen atom to which they are attached together form a 4- to 7-membered ring optionally having an additional heteroatom selected from NR^{x}, O and S(O)_{q}, and said ring is optionally substituted with 1 to 4 halogen atoms or a group selected from CO₂R^{a} and CONR^{a}R^{b};
R^{a} is H or C₁₋₆alkyl;
R^{b} and R^{c} are each independently selected from H, C₁₋₆alkyl, C₁₋₆haloalkyl, C₃₋₆cycloalkyl and C₁₋₆hydroxyalkyl; or
R^{b}, R^{c} and the nitrogen atom to which they are attached together form a 4- to 7-membered ring optionally having an additional heteroatom selected from NR^{x}, O and S(O)_{q}, said ring being optionally substituted with 1 to 4 halogen atoms;
R^{x} is selected from the group consisting of H, C₁₋₆alkyl, C₁₋₆haloalkyl, C₁₋₆alkylcarbonyl and S(O)₂C₁₋₆alkyl;
R^{y} is selected from the group consisting of (1) hydroxy, (2) oxo, (3) cyano, (4) halogen, (5) C₁₋₆alkyl, C₂₋₁₀alkenyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano, NR^{b}R^{c}, NHC(O)C₁₋₆alkyl and S(O)₂C₁₋₆alkyl, (6) CO₂R^{a}, (7) nitro, (8) NR^{b}R^{c}, (9) CONR^{b}R^{c}, (10) NR^{b}(CONR^{b}R^{c}), and (11) S(O)₂NR^{b}R^{c};
R^{z} is selected from the group consisting of (1) hydroxy, (2) oxo, (3) cyano, (4) halogen, (5) C₁₋₆alkyl, C₂₋₁₀alkenyl, C₁₋₆alkylcarbonyl, C₁₋₆alkoxy, C₁₋₆alkoxycarbonyl, each of which is optionally substituted with 1 to 5 groups independently selected from halogen, OR^{a}, cyano, NR^{b}R^{c}, NHC(O)C₁₋₆alkyl and S(O)₂C₁₋₆alkyl, (6) CO₂R^{a}, (7) nitro, (8) NR^{b}R^{c}, (9) CONR^{b}R^{c}, (10) NR^{b}(CONR^{b}R^{c}), (11) S(O)₂NR^{b}R^{c} and (12) a ring containing moiety which is: C₆₋₁₀aryl, C₆₋₁₀arylC₁₋₆alkyl, cycloalkyl, azetidinyl, a 5 or 6 membered saturated or partially saturated heterocyclic ring containing 1, 2 or 3 heteroatoms independently selected from N, O and S, a 5 membered heteroaromatic ring containing 1, 2, 3 or 4 heteroatoms independently selected from N, O and S, not more than one heteroatom of which is O or S, a 6 membered heteroaromatic ring containing 1, 2 or 3 nitrogen atoms or a 7-15 membered unsaturated, partially saturated or saturated heterocyclic ring containing 1, 2, 3 or 4 heteroatoms independently selected from N, O and S; any of which rings being optionally substituted 1 to 5 groups independently selected from the group consisting of hydroxy, cyano, halogen, C₁₋₆alkyl, C₁₋₆alkoxy, C₂₋₁₀alkenyl, C₁₋₆haloalkyl, amino, C₁₋₆ alkylamino and di(C₁₋₆alkyl)amino.

2. A compound of claim 1 wherein L is NR⁵.

3. A compound of claim 1 wherein L is O.

4. A compound of claim 1 wherein L is a bond.

5. A compound of claim 1 having the formula Ia: 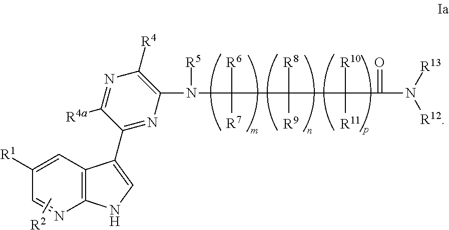
or a pharmaceutically acceptable salt thereof, wherein the variables are as defined in claim 1.

6. A compound of claim 5 wherein m and p are each 0, and n is 1 or 2.

7. A compound of claim 5 wherein m and p are each 0, n is 1, and one of R⁸ and R⁹ is C₁₋₄alkyl, and the other is H or C₁₋₄alkyl.

8. A compound of claim 5 wherein m and p are each 0, n is 1, and R⁸ and R⁹ together complete a C₃₋₆cycloalkyl.

9. A compound of claim 5 wherein m and p are each 0, n is 2 and two R⁸ groups on adjacent carbon atoms together form a bond, or the two R⁸ and two R⁹ groups on adjacent carbon atoms together form a cycloalkenyl, aryl or heteroaryl group.

10. A compound of claim 5 wherein R⁶ and R¹⁰ together complete a cycloalkyl, cycloalkenyl or heterocyclyl.

11. A compound of claim 5 wherein R⁵ and R¹⁰ together complete a heterocycle optionally substituted with one or two groups independently selected from halogen, OR^{a} and C₁₋₃ alkyl.

12. A compound of claim 1 wherein one of R¹² and R¹³ is H and the other is —CH₂CF₃.

13. A compound of claim 1 having the formula Ib: 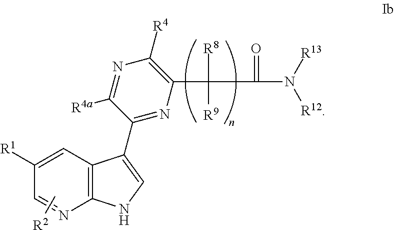
or a pharmaceutically acceptable salt thereof, wherein the variables are as defined in claim 1.

14. A compound of claim 1 having the formula Ic: 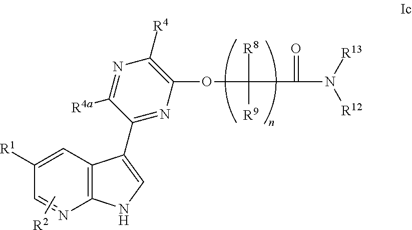
or a pharmaceutically acceptable salt thereof, wherein n is 1 or 2 and the other variables are as defined in claim 1.

15. A pharmaceutical composition comprising a compound of claim 1 and a pharmaceutically acceptable carrier.

16. A method for the treatment of Janus kinase mediated diseases which comprises administering to a patient in need thereof a therapeutically effective amount of a compound of claim 1.
